(19) United States
(12) Patent Application Publication
(10) Pub. No.: US 2020/0196845 A1
(43) Pub. Date: Jun. 25, 2020

(54) CAPSULE ENDOSCOPE SYSTEM, CAPSULE ENDOSCOPE, AND RECEIVING DEVICE

(71) Applicant: OLYMPUS CORPORATION, Tokyo (JP)

(72) Inventor: Takuto IKAI, Tokyo (JP)

(73) Assignee: OLYMPUS CORPORATION, Tokyo (JP)

(21) Appl. No.: 16/807,603

(22) Filed: Mar. 3, 2020

Related U.S. Application Data
(63) Continuation of application No. PCT/JP2018/021946, filed on Jun. 7, 2018.

(30) Foreign Application Priority Data
Sep. 15, 2017 (JP) 2017-178160

(51) Int. Cl. A61B 1/04; A61B 1/045; A61B 1/06; A61B 1/00

(52) U.S. Cl. CPC A61B 1/041; A61B 1/045; A61B 1/0661; A61B 2562/0219; A61B 1/00009; A61B 1/00027; A61B 2562/0247; A61B 1/00016

(57) ABSTRACT

A capsule endoscope system includes: a capsule endoscope configured to generate images by imaging a subject through irradiation of the subject with illumination light; and a processor including hardware, the processor being configured to determine, based on an image to be determined or associated information associated with the image to be determined, whether or not the image to be determined is suitable for control of operation of the capsule endoscope, the image being one of the images generated.

### CROSS-REFERENCE TO RELATED APPLICATION

This application is a continuation of PCT International Application No. PCT/JP2018/021946 filed on Jun. 7, 2018, which designates the United States, incorporated herein by reference, and which claims the benefit of priority from Japanese Patent Application No. 2017-178160, filed on Sep. 15, 2017, incorporated herein by reference.

### BACKGROUND

### 1. Technical Field

The present disclosure relates to: a capsule endoscope system where image data are acquired by use of a capsule endoscope introduced into a subject; the capsule endoscope; and a receiving device.

### 2. Related Art

In the related art, endoscopes have been in widespread use as medical observation devices, which are introduced into bodies of subjects, such as patients, and which are for observation of the interiors of the subjects. Furthermore, developed in recent years is a capsule endoscope that is a swallowable image acquisition device including, inside a capsule casing: an imaging device; and a communication device that wirelessly transmits, to the outside of a body, image data captured by this imaging device. The capsule endoscope has a function of moving inside organs, such as, for example, the esophagus, the stomach, and the small intestine, according to peristaltic movement of the organs, and sequentially capturing images therein, after being swallowed from the mouth of a patient for observation of the interior of the subject, until the capsule endoscope is naturally excreted from the subject.

While the capsule endoscope is moving in the subject, the image data captured by the capsule endoscope are sequentially transmitted to the outside of the body by wireless communication, and accumulated in a memory provided inside or outside a receiving device outside the body. A doctor or a nurse is able to: fetch the image data accumulated in the memory, into an information processing apparatus via a cradle having the receiving device inserted therein; and make diagnoses based on images displayed on a display of this information processing apparatus.

For efficient and accurate diagnoses to be performed, the number of images of the same subject image are desired to be reduced while many images of different subject images are desired to be acquired. For example, according to Japanese Patent No. 3952777, operation of a capsule endoscope is controlled by use of two images captured by the capsule endoscope at arbitrary times. Specifically, according to Japanese Patent No. 3952777, the frame rate is changed based on a result of comparison between the two images. For example, the moving velocity of the capsule endoscope is determined by comparison between the images, and the frame rate is changed based on a result of the determination. Redundant capturing of similar images is thereby able to be reduced.

### SUMMARY

In some embodiments, a capsule endoscope system includes: a capsule endoscope configured to generate images by imaging a subject through irradiation of the subject with illumination light; and a processor comprising hardware, the processor being configured to determine, based on an image to be determined or associated information associated with the image to be determined, whether or not the image to be determined is suitable for control of operation of the capsule endoscope, the image being one of the images generated.

In some embodiments, provided is a capsule endoscope configured to generate images by imaging a subject through irradiation of the subject with illumination light. The capsule endoscope includes: a processor including hardware, the processor being configured to determine, based on an image to be determined or associated information associated with the image to be determined, whether or not the image to be determined is suitable for control of operation of the capsule endoscope, the image to be determined being one of the images generated.

In some embodiments, provided is a receiving device configured to receive images wirelessly transmitted by a capsule endoscope configured to generate the images by imaging a subject through irradiation of the subject with illumination light. The receiving device includes: a processor comprising hardware, the processor being configured to determine, based on an image to be determined or associated information associated with the image to be determined, whether or not the image to be determined is suitable for control of operation of the capsule endoscope, the image to be determined being one of the images generated, and generate, from information obtained from the image that have been determined to be suitable, control information related to operation control of the capsule endoscope.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a capsule endoscope system according to a first embodiment of the present disclosure;

FIG. 2 is a block diagram illustrating a schematic configuration of the capsule endoscope system according to the first embodiment of the present disclosure;

FIG. 3 is a diagram for explanation of image data selection processing performed in the capsule endoscope system according to the first embodiment of the present disclosure;

FIG. 4 is a flow chart illustrating image data acquisition processing performed by the capsule endoscope system according to the first embodiment of the present disclosure;

FIG. 5 is a flow chart illustrating image data acquisition processing performed by a capsule endoscope system according to a first modified example of the first embodiment of the present disclosure;

FIG. 6 is a diagram illustrating a configuration of a capsule endoscope included in a capsule endoscope system according to a second modified example of the first embodiment of the present disclosure;

FIG. 7 is a diagram for explanation of image data selecting processing performed in the capsule endoscope system according to the second modified example of the first embodiment of the present disclosure;

FIG. 8 is a flow chart illustrating image data acquisition processing performed by a capsule endoscope system according to a third modified example of the first embodiment of the present disclosure;

FIG. 9 is a flow chart illustrating image data acquisition processing performed by a capsule endoscope system according to a fourth modified example of the first embodiment of the present disclosure;

FIG. 10 is a block diagram illustrating a schematic configuration of a capsule endoscope system according to a second embodiment of the present disclosure;

FIG. 11 is a flow chart illustrating image data acquisition processing performed by the capsule endoscope system according to the second embodiment of the present disclosure;

FIG. 12 is a flow chart illustrating image data acquisition processing performed by a capsule endoscope system according to a third embodiment of the present disclosure;

FIG. 13 is a flow chart illustrating image data acquisition processing performed by a capsule endoscope system according to a fourth embodiment of the present disclosure;

FIG. 14 is a flow chart illustrating image data acquisition processing performed by a capsule endoscope system according to a fifth embodiment of the present disclosure;

FIG. 15 is a block diagram illustrating a schematic configuration of a capsule endoscope system according to a sixth embodiment of the present disclosure;

FIG. 16 is a flow chart illustrating image data acquisition processing performed by the capsule endoscope system according to the sixth embodiment of the present disclosure;

FIG. 17 is a block diagram illustrating a schematic configuration of a capsule endoscope system according to a seventh embodiment of the present disclosure;

FIG. 18 is a flow chart illustrating image data acquisition processing performed by the capsule endoscope system according to the seventh embodiment of the present disclosure; and

FIG. 19 is a flow chart illustrating image data acquisition processing performed by a capsule endoscope system according to a modified example of the seventh embodiment of the present disclosure.

### DETAILED DESCRIPTION

Described hereinafter as embodiments according to the present disclosure are capsule endoscope systems where medical capsule endoscopes are used. The same reference sign will be assigned to parts that are the same, throughout the drawings. Furthermore, it needs to be noted that the drawings are schematic, and a relation between a thickness and a width of each member and proportions among the members may be different from the actual relation and proportions.

### First Embodiment

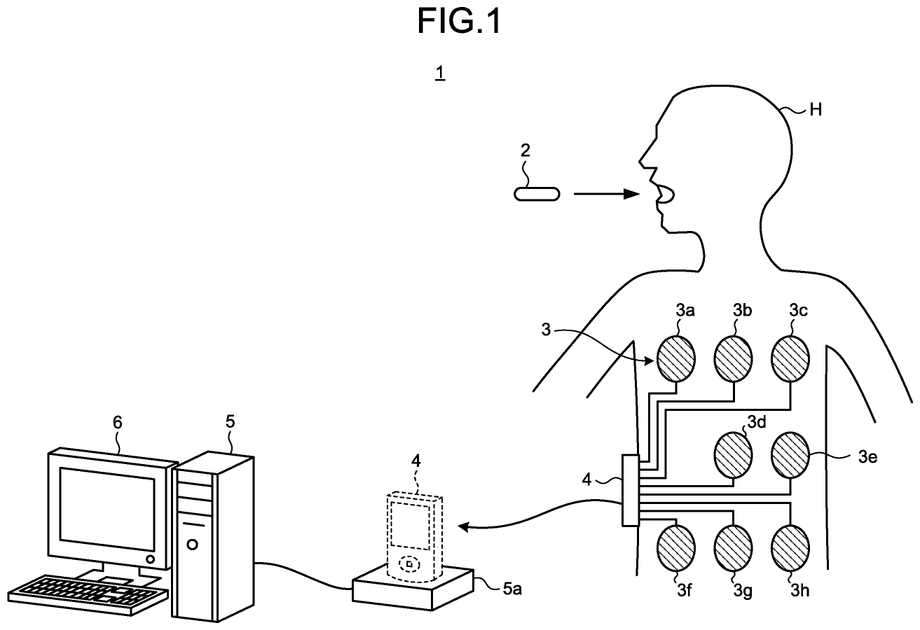

FIG. 1 is a schematic diagram illustrating a schematic configuration of a capsule endoscope system according to a first embodiment of the present disclosure. As illustrated in FIG. 1, a capsule endoscope system 1 according to the first embodiment includes: a capsule endoscope 2, which is an image acquisition device that generates image data by being introduced into a subject H and imaging the interior of the subject H and transmits the image data via a radio wave by superimposing the image data on a wireless signal; a receiving device 4 that receives the wireless signal transmitted from the capsule endoscope 2 via a receiving antenna unit 3 including plural receiving antennas 3a to 3h attached to the subject H; and a processing device 5 that fetches the image data generated by the capsule endoscope 2 from the receiving device 4 via a cradle 5a, processes the image data, and generates an image of the interior of the subject H. The image generated by the processing device 5 is, for example, output from a display device 6 by being displayed thereon.

After being swallowed by the subject H, the capsule endoscope 2 sequentially captures images of parts of the living body (the esophagus, the stomach, the small intestine, and the large intestine) at preset reference cycles (for example, at 0.5-second cycles), while moving in the digestive tract of the subject H, according to peristaltic movement of the organs. The image data and associated information acquired by this imaging operation are sequentially transmitted wirelessly to the receiving device 4.

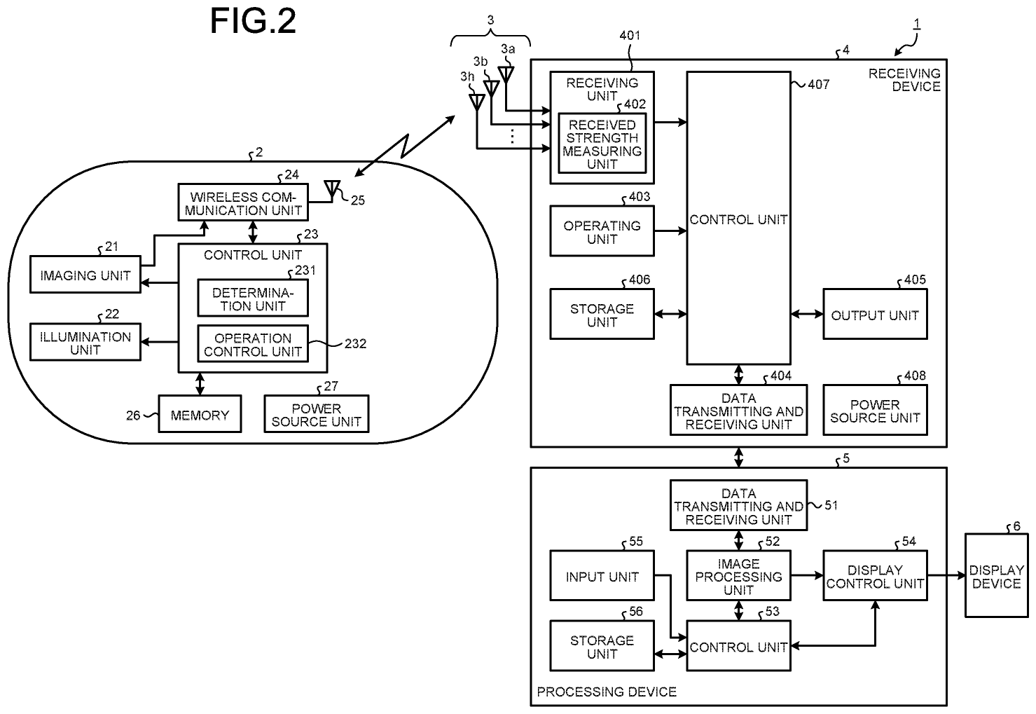

FIG. 2 is a block diagram illustrating a schematic configuration of the capsule endoscope system according to the first embodiment of the present disclosure. The capsule endoscope 2 includes an imaging unit 21, an illumination unit 22, a control unit 23, a wireless communication unit 24, an antenna 25, a memory 26, and a power source unit 27. The capsule endoscope 2 is a device having the above described built-in parts in a capsule-shaped casing having a size swallowable by the subject H.

The imaging unit 21 includes, for example: an imaging element that generates image data resulting from imaging of the interior of the subject H, from an optical image formed on a light receiving surface thereof, and outputs the image data; and an optical system, such as an objective lens, which is arranged on a light receiving surface side of the imaging element. The imaging element is formed of a charge coupled device (CCD) imaging element or a complementary metal oxide semiconductor (CMOS) imaging element, includes plural pixels that receive light from the subject H, the plural pixels being arranged in a matrix, and generates image data by photoelectrically converting the light received by the pixels. For the plural pixels arranged in a matrix, the imaging unit 21 reads out pixel values per horizontal line and generates image data including plural sets of line data having a synchronization signal allocated to each horizontal line.

The illumination unit 22 is formed of a white LED that generates white light serving as illumination light. Instead of being formed of a white LED, the illumination unit 22 may be configured to generate white light by combining light of plural LEDs or laser light sources that have different emission wavelength bands, or may be configured by use of a xenon lamp or a halogen lamp.

The control unit 23 controls operation and processing of each part forming the capsule endoscope 2. When, for example, the imaging unit 21 performs imaging processing; the control unit 23 controls the imaging unit 21 such that the imaging unit 21 executes exposure and reading processing for the imaging element, and controls the illumination unit 22 such that the illumination unit 22 emits the illumination light according to exposure timing of the imaging unit 21. Furthermore, the control unit 23 determines, from pixel values (luminance values) of the image data captured by the imaging unit 21, a light emission time period of the illumination unit 22 for the next imaging, and controls the illumination unit 22 such that the illumination unit 22 emits illumination light over the determined light emission time period. Accordingly, the time period of light emission by the illumination unit 22 is controlled by the control unit 23, based on the image data captured, and the light emission time period may change every time imaging is performed. The control unit 23 is formed by use of a general-purpose processor, such as a central processing unit (CPU), or a special-purpose processor, such as an arithmetic circuit that executes a specific function, like an application specific integrated circuit (ASIC).

Furthermore, the control unit 23 includes: a determination unit 231 that determines usability of image data for control of frame rate of the imaging unit 21; and an operation control unit 232 that controls frame rate of the imaging unit 21, based on a result of the determination by the determination unit 231.

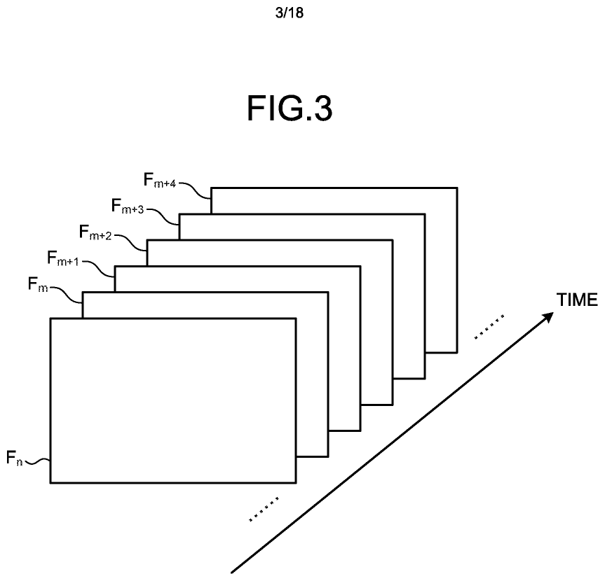

FIG. 3 is a diagram for explanation of image data selection processing performed in the capsule endoscope system according to the first embodiment of the present disclosure. The imaging unit 21 performs imaging processing at a preset frame rate and sequentially generates image data (FIG. 3 illustrating images Fₙ, Fₘ, and Fₘ₊₁ to Fₘ₊₄ corresponding to the image data, where n and m are both natural numbers and n<m). The determination unit 231 selects two images (image data) that control the frame rate, by determining whether or not the two images are suitable as images for use in control of the frame rate. For example, after selecting a first set of image data that is a set of image data corresponding to the n-th image Fₙ from plural sets of image data, the determination unit 231 selects a second set of image data from the m-th and (m+1)-th to (m+4)-th images Fₘ and Fₘ₊₁ to Fₘ₊₄, the second set of image data being a set of image data that is chronologically after the first set of image data selected. According to the first embodiment, based on the light emission time period of the illumination unit 22 for imaging of each set of image data, the determination unit 231 selects image data suitable for control of the frame rate. After a first set of image data is selected, a second set of image data may be selected from sets of image data that are chronologically before the first set of image data.

The operation control unit 232 calculates the degree of similarity between the two sets of image data selected by the determination unit 231 and compares this degree of similarity with a threshold that has been stored in the memory 26 beforehand. According to a result of the comparison between the degree of similarity and the threshold, the operation control unit 232 sets a frame rate of the imaging unit 21. This frame rate set is a value indicating an interval of acquisition of image data by the imaging unit 21. The imaging unit 21 executes imaging processing, based on the frame rate set by the operation control unit 232.

The wireless communication unit 24 processes image data output from the imaging unit 21. The wireless communication unit 24 acquires digital image data by performing A/D conversion and predetermined signal processing on the image data output from the imaging unit 21, superimposes the digital image data, together with associated information, on a wireless signal, and transmits the superimposed digital image data to outside, from the antenna 25. The associated information includes identification information (for example, a serial number) that has been allocated for identification of the individuality of the capsule endoscope 2.

The memory 26 stores therein an execution program and a control program for execution of various types of operation, and parameters, such as thresholds. Furthermore, the memory 26 may temporarily store therein image data that have been signal-processed by the wireless communication unit 24. The memory 26 is formed of a random access memory (RAM) and/or a read only memory (ROM).

The power source unit 27 includes: a battery formed of, for example, a button cell; a power source circuit that boosts electric power from the battery; and a power source switch that switches the power source unit 27 between an on-state and an off-state; and the power source unit 27 supplies electric power to each unit in the capsule endoscope 2 after the power source switch is turned on. The power source switch is formed of, for example, a reed switch that is switched over between an on-state and an off-state by external magnetic force, and is switched to the on-state by application of magnetic force from outside to the capsule endoscope 2 before use of the capsule endoscope 2 (that is, before the subject H swallows the capsule endoscope 2).

The receiving device 4 includes a receiving unit 401, a received strength measuring unit 402, an operating unit 403, a data transmitting and receiving unit 404, an output unit 405, a storage unit 406, a control unit 407, and a power source unit 408 that supplies electric power to each of these units.

The receiving unit 401 receives, via the receiving antenna unit 3 having the plural (eight in FIG. 1) receiving antennas 3a to 3h, image data and associated information wirelessly transmitted from the capsule endoscope 2. Each of the receiving antennas 3a to 3h is realized by use of, for example, a loop antenna or a dipole antenna, and is arranged at a predetermined position on an outer surface of the body of the subject H. The receiving unit 401 includes the received strength measuring unit 402 that measures received signal strength indicators (RSSIs) of wireless signals received by the receiving antennas 3a to 3h. Based on the received signal strength indicators measured by the received strength measuring unit 402, the receiving unit 401 selects an antenna having the highest received signal strength indicator, from the receiving antennas 3a to 3h, and receives the wireless signal received by that antenna selected. Furthermore, the receiving unit 401 is formed of, for example, a processor, such as a CPU or an ASIC, and performs predetermined signal processing, such as A/D conversion, on the received image data.

The received strength measuring unit 402 measures a received signal strength indicator for a wireless signal received by the receiving unit 401, for each of the receiving antennas 3a to 3h. All of the received signal strength indicators that have been measured may be stored in the storage unit 406, in association with the image data received by the receiving unit 401.

The operating unit 403 is an input device that is used when a user inputs various types of setting information and instruction information to the receiving device 4. The operating unit 403 is, for example, a switch or a button provided on an operating panel of the receiving device 4.

The data transmitting and receiving unit 404 transmits image data and associated information that have been stored in the storage unit 406, to the processing device 5, when the data transmitting and receiving unit 404 is connected to the processing device 5 in a state where the data transmitting and receiving unit 404 is able to communicate with the processing device 5. The data transmitting and receiving unit 404 is formed of a communication interface, such as a LAN.

The output unit 405 is configured to display an image, output sound or light, and generate vibration. The output unit 405 is configured to display a notification image according to an interference level and generate sound, light, and/or vibration. The output unit 405 is formed of at least one selected from a group including: a display, such as a liquid crystal display or an organic EL display; a speaker; a light source; and a vibration generator, such as a vibrating motor.

The storage unit 406 stores therein: a program for causing the receiving device 4 to operate and execute various functions; and image data acquired by the capsule endoscope 2. The storage unit 406 is formed of a RAM and/or a ROM.

The control unit 407 controls each unit forming the receiving device 4. The control unit 407 is formed by use of: a general-purpose processor, such as a CPU; or a special-purpose processor, such as an arithmetic circuit that executes a specific function, like an ASIC.

This receiving device 4 is attached to and carried by the subject H while imaging is being performed by the capsule endoscope 2, for example, while the capsule endoscope 2 is passing through the digestive tract after being swallowed by the subject H until the capsule endoscope 2 is excreted. The receiving device 4 stores image data received via the receiving antenna unit 3 during this imaging, into the storage unit 406.

After the imaging by the capsule endoscope 2 is finished, the receiving device 4 is removed from the subject H and is set in the cradle 5a (as seen FIG. 1) connected to the processing device 5. As a result, the receiving device 4 is connected to the processing device 5 in a state of being communicable with the processing device 5 and transfers (downloads) the image data and associated information stored in the storage unit 406, to the processing device 5.

The processing device 5 is formed by use of, for example, a work station having the display device 6, such as a liquid crystal display. The processing device 5 includes a data transmitting and receiving unit 51, an image processing unit 52, a control unit 53 that integrally controls the respective units, a display control unit 54, an input unit 55, and a storage unit 56.

The data transmitting and receiving unit 51 is an interface that is connectable to a USB, or a communication line, such as a wired LAN or a wireless LAN, and includes a USB port and a LAN port. According to the first embodiment, the data transmitting and receiving unit 51 is connected to the receiving device 4 via the cradle 5a connected to the USB port and transmits and receives data to and from the receiving device 4.

By reading a predetermined program stored in the storage unit 56 described later, the image processing unit 52 executes predetermined image processing for generating an in-vivo image corresponding to image data input from the data transmitting and receiving unit 51 or image data stored in the storage unit 56. The image processing unit 52 is realized by a processor, such as a CPU or an ASIC.

By reading various programs stored in the storage unit 56, the control unit 53 executes, based on signals input via the input unit 55 and image data input from the data transmitting and receiving unit 51, transfer of instructions and data to the respective units forming the processing device 5, and integrally controls the overall operation of the processing device 5. The control unit 53 is realized by: a general-purpose processor, such as a CPU; or a special-purpose processor, such as an arithmetic circuit that executes a specific functions, like an ASIC.

After performing predetermined processing, such as data thinning and/or gradation processing, on an image generated in the image processing unit 52, the predetermined processing corresponding to an image display range in the display device 6, the display control unit 54 causes the display device 6 to output the processed image by displaying thereon the processed image. The display control unit 54 is formed of, for example, a processor, such as a CPU or an ASIC.

The input unit 55 receives input of information or a command according to operation by a user. The input unit 55 is realized by an input device, such as, for example: a keyboard and a mouse; a touch panel; or various switches.

The storage unit 56 stores therein a program for causing the processing device 5 to operate and execute various functions, various kinds of information used during the execution of the program, image data and associated information acquired via the receiving device 4, and in-vivo images generated by the image processing unit 52. The storage unit 56 is realized by: a semiconductor memory, such as a flash memory, a RAM, or a ROM; or a recording medium, such as an HDD, an MO, a CD-R, or a DVD-R, and a drive device that drives the recording medium.

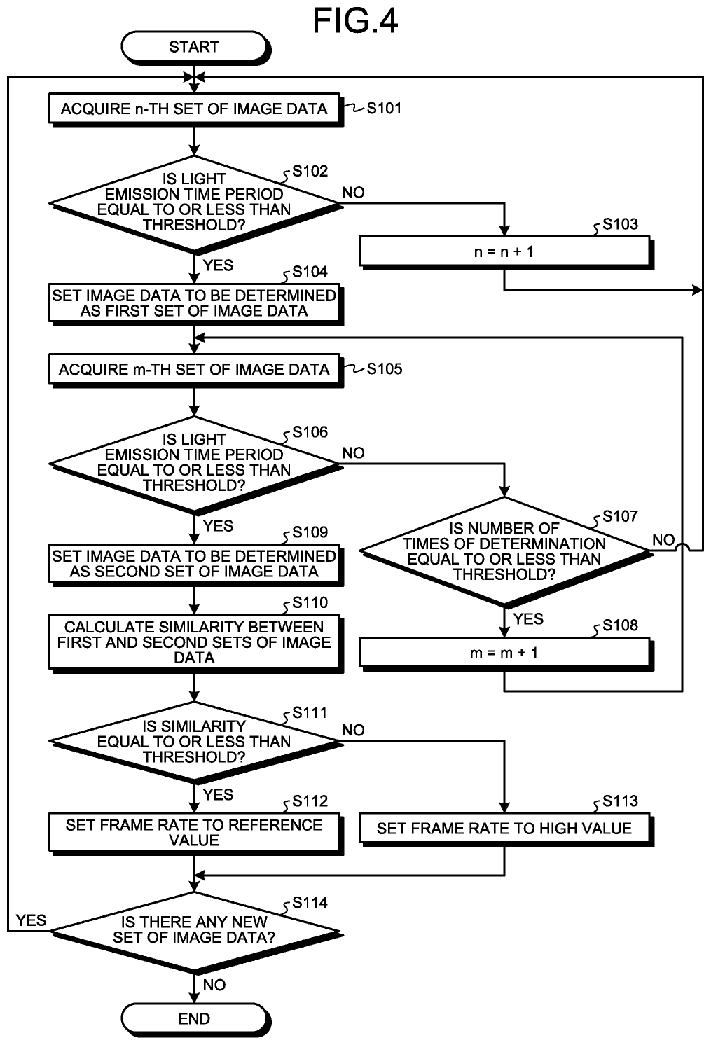

Described next is image data acquisition processing executed by the capsule endoscope 2. FIG. 4 is a flow chart illustrating the image data acquisition processing performed by the capsule endoscope system according to the first embodiment of the present disclosure. Hereinafter, description will be made on the assumption that each unit operates under control of the control unit 23.

The control unit 23 causes the imaging unit 21 to start imaging processing and acquires an n-th set of image data (where n=1 immediately after the start) (Step S101).

At Step S102 subsequent to Step S101, the determination unit 231 determines whether or not a light emission time period of illumination light for the capturing of the n-th set of image data acquired at Step S101 is equal to or less than a threshold. If the determination unit 231 determines that the light emission time period is greater than the threshold (Step S102: No), the control unit 23 proceeds to Step S103.

At Step S103, the control unit 23 increments the counter n by 1 and returns to Step S101. This means that the set of image data to be determined is changed to a set of image data of a frame with a later acquisition time.

On the contrary, at Step S102, if the determination unit 231 determines that the light emission time period is equal to or less than the threshold (Step S102: Yes), the processing is advanced to Step S104.

At Step S104, the determination unit 231 sets this n-th set of image data as one (hereinafter referred to as a first set of image data) of two sets of image data that are selected.

At Step S105 subsequent to Step S104, the control unit 23 acquires an m-th set of image data (where m>n).

At Steps S106 to S109 subsequent to Step S105, by determining whether a light emission time period of illumination light for the capturing of the m-th set of image data acquired at Step S105 is equal to or less than the threshold, the determination unit 231 performs setting of the other one (hereinafter, referred to a second set of image data) of the two sets of image data. If the determination unit 231 determines that the light emission time period is greater than the threshold (Step S106: No), the control unit 23 proceeds to Step S107.

At Step S107, the determination unit 231 determines whether or not the number of times for which the determination for the setting of the second set of image data has been performed is equal to or less than a threshold that is a preset number of times. If the determination unit 231 determines that the number of times of determination is greater than the threshold (Step S107: No), the processing is returned to Step S101 and the processing is redone from the setting of the first set of image data. On the contrary, if the determination unit 231 determines that the number of times of determination is equal to or less than the threshold (Step S107: Yes), the control unit 23 proceeds to Step S108.

At Step S108, the control unit 23 increments the counter m by 1 and returns to Step S105. This means that the set of image data to be determined is changed to a set of image data of a frame with a later acquisition time.

On the contrary, at Step S106, if the determination unit 231 determines that the light emission time period is equal to or less than the threshold (Step S106: Yes), the processing is advanced to Step S109.

At Step S109, the determination unit 231 sets this m-th set of image data as the second set of image data.

At Step S110 subsequent to Step S109, the operation control unit 232 calculates the degree of similarity between images of the first set of image data and second set of image data. The degree of similarity calculated by the operation control unit 232 is calculated by use of a known method, such as Sum of Squared Differences (SSD), Sum of Absolute Differences (SAD), Normalized Cross-Correlation (NCC), or Zero-mean Normalized Cross-Correlation (ZNCC). Difference between pixel values (luminance values) of corresponding pixels of the images may be calculated as the degree of similarity. Described according to the first embodiment is an example where the SSD is calculated as the degree of similarity.

At Step S111 subsequent to Step S110, the operation control unit 232 determines whether or not the degree of similarity calculated is equal to or less than a threshold. If the operation control unit 232 determines that the degree of similarity is equal to or less than the threshold (Step S111: Yes), the processing is advanced to Step S112. If the degree of similarity is equal to or less than the threshold, it is able to be determined that movement of the subject between the images is small and similar subject images have been captured therein.

At Step S112, the operation control unit 232 sets the frame rate to a reference value. This reference value is a value where the imaging unit 21 performs imaging at reference cycles (for example, 0.5-second cycles) as described above. After setting the frame rate to the reference value, the operation control unit 232 controls the imaging unit 21 such that the imaging unit 21 executes imaging processing at intervals according to this reference value.

On the contrary, at Step S111, if the operation control unit 232 determines that the degree of similarity is greater than the threshold (Step S111: No), the processing is advanced to Step S113. If the degree of similarity is greater than the threshold, it is able to be determined that movement of the subject between the images is large and different subject images have been captured therein.

At Step S113, the operation control unit 232 sets the frame rate to a high value. This high value is a value where the imaging unit 21 performs imaging at cycles (for example, 0.25-second cycles) shorter than the reference cycles (for example, 0.5-second cycles) described above. After setting the frame rate to the high value, the operation control unit 232 controls the imaging unit 21 such that the imaging unit 21 executes imaging processing at intervals according to this high value.

After setting of the frame rate by the operation control unit 232, the control unit 23 determines whether or not a new set of image data has been generated (Step S114). If the control unit 23 determines that a new set of image data has been generated (Step S114: Yes), the control unit 23 returns to Step S101 and repeats the above described processing by treating the latest set of image data at that time point as the n-th set of image data. On the contrary, if a new set of image data has not been generated (Step S114: No), the control unit 23 ends the image data acquisition processing.

According to the first embodiment described above, whether or not an image is suitable as an image used in setting of a frame rate is determined from a light emission time period of illumination light for capturing of a set of image data, the degree of similarity between two images determined to be suitable is calculated, and the frame rate is set based on a result of the calculation. According to the first embodiment, because operation control is performed by use of images suitable for use in control, the images being from images captured by a capsule endoscope, operation of the capsule endoscope is able to be controlled adequately.

According to the first embodiment, a first set of image data and a second set of image data are selected based on light emission time periods, but a set of image data may be selected based on a light emission intensity, which is used, in addition to a light emission time period, for control of light emission quantity of a capsule endoscope. If the selection is performed based on the light emission time period, the light emission intensity is constant, and if the selection is performed based on the light emission intensity, the light emission time period is constant. As described above, according to the first embodiment, selection of a set of image data is performed by use of a light emission time period or a light emission intensity, which is used in control of light emission quantity.

Moreover, according to the above description of the first embodiment, in the control of the frame rate, if the degree of similarity is greater than the threshold, the frame rate is changed from the reference value to a high value, but a high value may be set as the reference value and if the degree of similarity is equal to or less than the threshold, the frame rate may be set to a low value indicating cycles longer than the reference cycles.

According to the first embodiment, the threshold is set on the assumption that the degree of similarity is the SSD, but if another value (for example, an NCC) is calculated as the degree of similarity, the value of the threshold and the magnitude correlation used in the determination are changed depending on the relation between the magnitude of the value and the degree of similarity, needless to say.

First Modified Example of First Embodiment

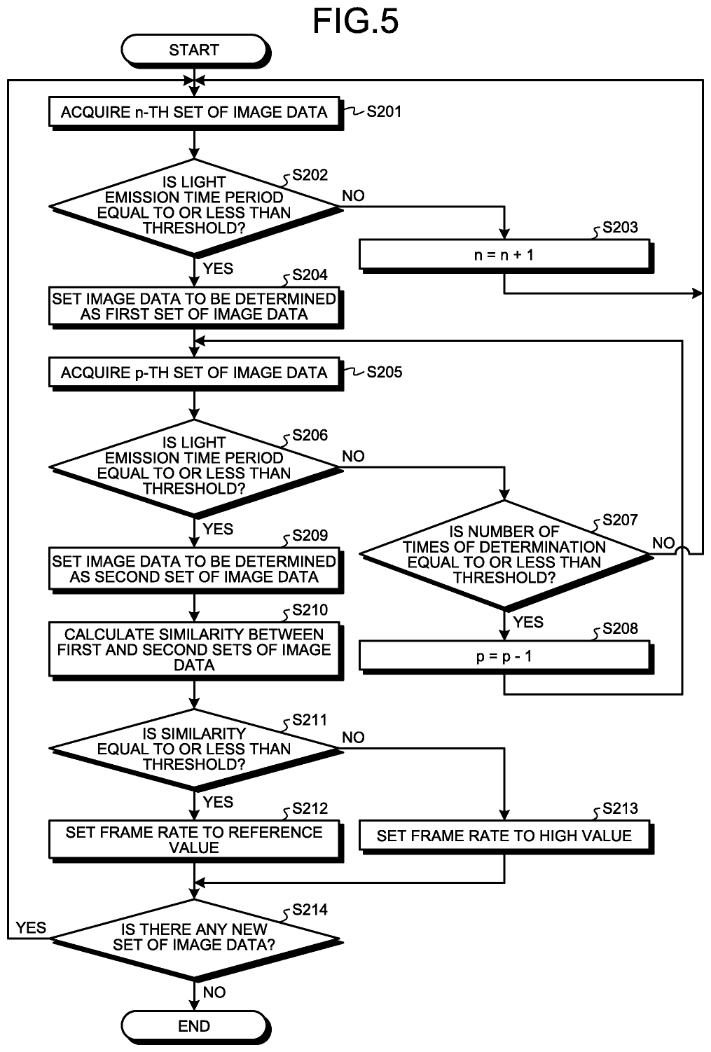

Described next is a first modified example of the first embodiment of the present disclosure. FIG. 5 is a flow chart illustrating image data acquisition processing performed by a capsule endoscope system according to the first modified example of the first embodiment of the present disclosure. The capsule endoscope system according to the first modified example is similar to the capsule endoscope system 1 described above. Described hereinafter by reference to FIG. 5 is processing different from the processing according to the first embodiment described above. In this first modified example, when a second set of image data is set, determination processing is performed, the determination processing being where the second set of image data is selected in order from a set of image data chronologically apart from a first set of image data. In this first modified example, description will be made on the assumption that sets of image data corresponding to a preset number of frames including the latest frame have been stored in the memory 26, the sets of image data being from sets of image data generated by the imaging unit 21.

In this first modified example, similarly to Steps S101 to S104 illustrated in FIG. 4, the control unit 23 sets an n-th set of image data as a first set of image data (Steps S201 to S204).

At Step S205 subsequent to Step S204, the control unit 23 acquires a p-th set of image data (where p>n). The counter p is a value resulting from addition of the maximum value (a threshold) of the number of times of determination to the counter n.

At Steps S206 to S209 subsequent to Step S205, by determining whether or not a light emission time period of illumination light for the capturing of the p-th set of image data acquired at Step S205 is equal to or less than the threshold, the determination unit 231 sets a second set of image data. If the determination unit 231 determines that the light emission time period is greater than the threshold (Step S206: No), the control unit 23 proceeds to Step S207.

At Step S207, the determination unit 231 determines whether or not the number of times for which the determination for the setting of the second set of image data has been performed is equal to or less than a threshold that is a preset number of times. If the determination unit 231 determines that the number of times of determination is greater than the threshold (Step S207: No), the processing is returned to Step S201 and the processing is redone from the setting of the first set of image data. On the contrary, if the determination unit 231 determines that the number of times of determination is equal to or less than the threshold (Step S207: Yes), the control unit 23 proceeds to Step S208.

At Step S208, the control unit 23 decrements the counter p by 1 and returns to Step S205. This means that the set of image data to be determined is changed to a set of image data of a frame with an older acquisition time.

On the contrary, at Step S206, if the determination unit 231 determines that the light emission time period is equal to or less than the threshold (Step S206: Yes), the processing is advanced to Step S209.

At Step S209, the determination unit 231 sets this m-th set of image data as the second set of image data.

At Steps S210 to S213 subsequent to Step S209, the operation control unit 232 calculates the degree of similarity and sets a frame rate, similarly to Steps S110 to S113 illustrated in FIG. 4.

After setting of the frame rate by the operation control unit 232, the control unit 23 determines whether or not a new set of image data has been generated (Step S214). If the control unit 23 determines that a new set of image data has been generated (Step S214: Yes), the control unit 23 returns to Step S201 and repeats the above described processing by treating the latest set of image data at that time point as the n-th set of image data. On the contrary, if a new set of image data has not been generated (Step S214: No), the control unit 23 ends the image data acquisition processing.

According to the first modified example described above, effects similar to those of the first embodiment described above are able to be achieved, and additionally, because whether or not a second set of image data is suitable for control of the frame rate is determined in order from the latest one, the frame rate is able to be controlled by calculation of the degree of similarity between images through comparison with a set of image data closer to the latest set of image data.

In the first modified example described above, an image with the least blurring of the subject may be extracted from image data stored in the memory 26, and the operation control unit 232 may control the frame rate by using this extracted image. The amount of blurring of the subject in each image may be calculated by use of a known method.

Second Modified Example of First Embodiment

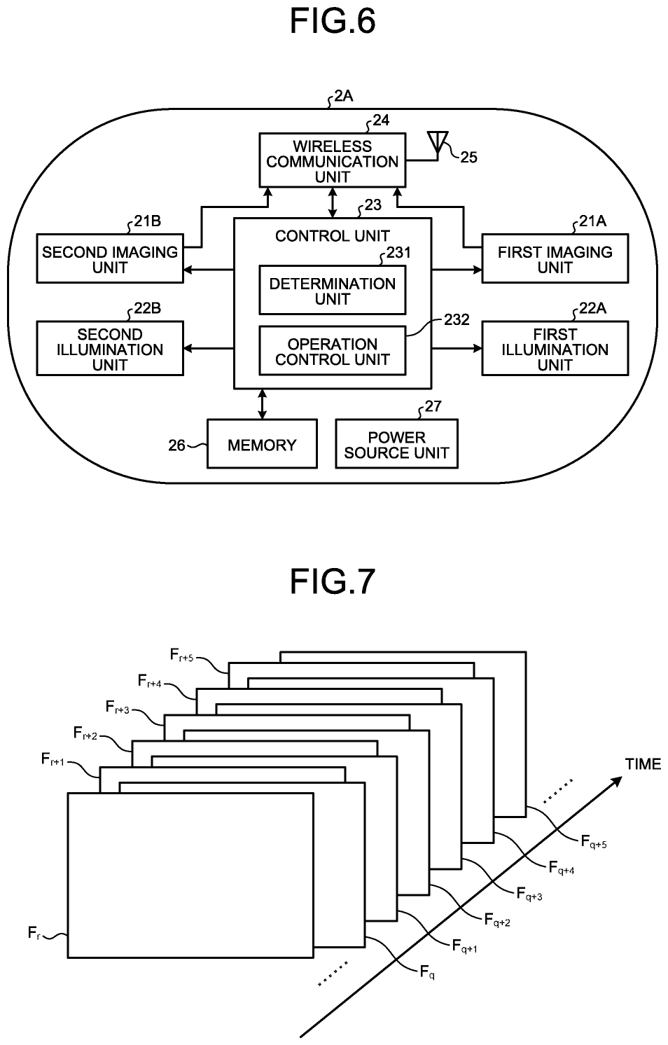

Described next is a second modified example of the first embodiment of the present disclosure. FIG. 6 is a diagram illustrating a configuration of a capsule endoscope included in a capsule endoscope system according to the second modified example of the first embodiment of the present disclosure. The capsule endoscope system according to the second modified example is different from the above described capsule endoscope system 1 in that the capsule endoscope system according to the second modified example includes a capsule endoscope 2A instead of the capsule endoscope 2. The rest of the configuration of the capsule endoscope system according to the second modified example is similar to that of the capsule endoscope system 1. Described hereinafter by reference to FIG. 6 and FIG. 7 are the configuration and processing different from those according to the first embodiment described above.

The capsule endoscope 2A according to the second modified example includes a first imaging unit 21A, a second imaging unit 21B, a first illumination unit 22A, a second illumination unit 22B, a control unit 23, a wireless communication unit 24, an antenna 25, a memory 26, and a power source unit 27. That is, the capsule endoscope 2A is different from the capsule endoscope 2 in that the capsule endoscope 2A includes the first imaging unit 21A, the second imaging unit 21B, the first illumination unit 22A, and the second illumination unit 22B, instead of the imaging unit 21 and the illumination unit 22. Described hereinafter are just the first imaging unit 21A, the second imaging unit 21B, the first illumination unit 22A, and the second illumination unit 22B, which are components different from those of the capsule endoscope 2.

Each of the first imaging unit 21A and the second imaging unit 21B includes, for example: an imaging element that generates image data representing the interior of the subject H from an optical image formed on a light receiving surface and outputs the image data; and an optical system, such as an objective lens, which is located on a light receiving surface side of the imaging element. The imaging element is formed of a CCD imaging element or a CMOS imaging element, includes plural pixels that receive light from the subject H, the plural pixels being arranged in a matrix, and generates the image data by photoelectrically converting the light received by the pixels.

The first imaging unit 21A and the second imaging unit 21B capture images in imaging directions different from each other. The capsule endoscope 2A is a twin-lens type capsule medical device that capture images forward and backward along a longitudinal direction of the capsule endoscope 2A, and in this second modified example, optical axes of the first imaging unit 21A and second imaging unit 21B are substantially parallel to or substantially coincides with a central axis along the longitudinal direction of the capsule endoscope 2A. Furthermore, the imaging directions of the first imaging unit 21A and the second imaging unit 21B are opposite to each other.

FIG. 7 is a diagram for explanation of image data selection processing performed in the capsule endoscope system according to the second modified example of the first embodiment of the present disclosure. The first imaging unit 21A and the second imaging unit 21B execute imaging processing alternately, under control of the control unit 23. As illustrated in FIG. 7, images F_{q} and F_{q+1} to F_{q+5} corresponding to image data generated by the first imaging unit 21A and images Fᵣ and Fᵣ₊₁ to Fᵣ₊₅ corresponding to image data generated by the second imaging unit 21B are thereby generated alternately one by one.

Each of the first illumination unit 22A and the second illumination unit 22B is formed of a white LED that generates white light serving as illumination light. Under control of the control unit 23, each of the first illumination unit 22A and the second illumination unit 22B emits illumination light in a light emission time period (or with a light intensity) that has been set.

When controlling frame rate of imaging units (the first imaging unit 21A and the second imaging unit 21B), the control unit 23 selects image data for use in setting of the frame rate by referring alternately to a pair of sets of image data acquired by the first imaging unit 21A and a pair of sets of image data acquired by the second imaging unit 21B. Specifically, for a pair of sets of image data (for example, the images F_{q} and F_{q+1} illustrated in FIG. 7) acquired by the first imaging unit 21A, the determination unit 231 compares light emission time periods for the capturing of these sets of image data with a threshold and determines whether or not these sets of image data are suitable as sets of image data for use in control. If the determination unit 231 determines that the sets of image data are suitable, the determination unit 231 sets the sets of image data corresponding to the images F_{q} and F_{q+1} as a first set of image data and a second set of image data and outputs the first set of image data and the second set of image data to the operation control unit 232. On the contrary, if the determination unit 231 determines that the images F_{q} and F_{q+1} are not suitable for use in control, the determination unit 231 compares, for a pair of sets of image data acquired by the second imaging unit 21B (for example, the images Fᵣ and Fᵣ₊₁ illustrated in FIG. 7), light emission time periods for the acquisition of these sets of image data with a threshold, and determines whether or not these images are suitable as images for use in control. The determination unit 231 thus selects sets of image data to be used in control (a first set of image data and a second set of image data) by repeating the determination in order from the images F_{q} and F_{q+1}, the images Fᵣ and Fᵣ₊₁, the images F_{q+1} and F_{q+2}, the images Fᵣ₊₁ and Fᵣ₊₂, and so on, until the determination unit 231 sets the first set of image data and the second set of image data.

By using the first set of image data and second set of image data selected by the determination unit 231, the operation control unit 232 sets the frame rate through calculation of the degree of similarity and comparison between the degree of similarity and a threshold, as described above. The operation control unit 232 may control only the imaging unit that has generated the sets of image data used in the determination, or may control the frame rate of both the first imaging unit 21A and the second imaging unit 21B.

In the second modified example, even if the capsule endoscope 2A includes the two imaging units, effects similar to those of the first embodiment described above are able to be achieved.

In the second modified example, one of the imaging units (for example, the first imaging unit 21A) may be set as an imaging unit to be subjected to determination by the determination unit 231, and processing similar to that according to the first embodiment described above may be performed on sets of image data generated by this first imaging unit 21A.

Third Modified Example of First Embodiment

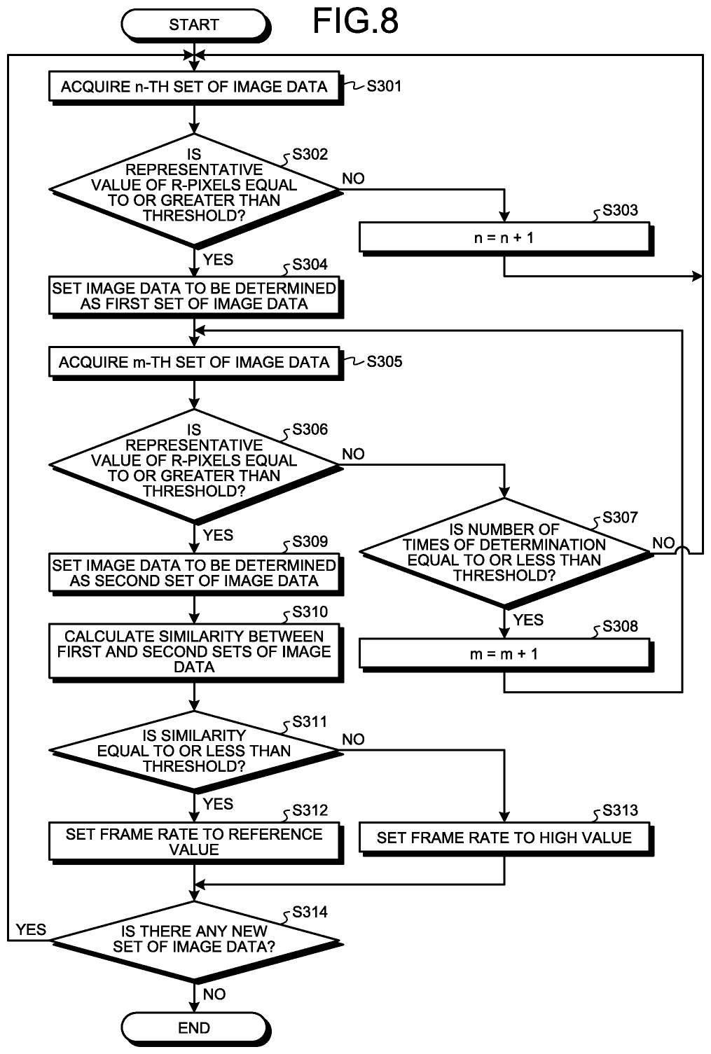

Described next is a third modified example of the first embodiment of the present disclosure. FIG. 8 is a flow chart illustrating image data acquisition processing performed by a capsule endoscope system according to a third modified example of the first embodiment of the present disclosure. The capsule endoscope system according to the third modified example is similar to the capsule endoscope system 1 described above. Described hereinafter by reference to FIG. 8 is processing different from the processing according to the first embodiment described above. In the third modified example, each pixel of an imaging element is provided with a color filter of any one of red (R), green (G), and blue (B), and receives light transmitted through that color filter. Hereinafter, a pixel that is provided with a red color filter and outputs a pixel value according to red light will be referred to as an R-pixel. Similarly, a pixel that is provided with a green color filter and outputs a pixel value according to green light will be referred to as a G-pixel, and a pixel that is provided with a blue color filter and outputs a pixel value according to blue light will be referred to as a B-pixel.

The control unit 23 causes the imaging unit 21 to start imaging processing and acquires an n-th set of image data (Step S301).

At Step S302 subsequent to Step S301, the determination unit 231 calculates a representative value of pixel values of R-pixels from the n-th set of image data acquired at Step S301 and determines whether or not the representative value of these R-pixels is equal to or greater than a threshold. The representative value of the R-pixels is, for example, the average value or the total of the pixel values of the R-pixels. If the control unit 23 determines that the representative value of the R-pixels is less than the threshold (Step S302: No), the control unit 23 proceeds to Step S303. An image having R-pixels with a representative value less than the threshold is able to be determined as an image having many residues and/or bubbles.

At Step S303, the control unit 23 increments the counter n by 1 and returns to Step S301.

On the contrary, at Step S302, if the determination unit 231 determines that the representative value of the R-pixels is equal to or greater than the threshold (Step S302: Yes), the processing is advanced to Step S304.

At Step S304, the determination unit 231 sets this n-th set of image data as a first set of image data.

At Step S305 subsequent to Step S304, the control unit 23 acquires an m-th set of image data (where m>n).

At Steps S306 to S309 subsequent to Step S305, the determination unit 231 determines whether or not a representative value of R-pixels in the m-th set of image data acquired at Step S305 is equal to or greater than the threshold. If the determination unit 231 determines that the representative value of the R-pixels is less than the threshold (Step S306: No), the control unit 23 proceeds to Step S307.

At Step S307, the determination unit 231 determines whether or not the number of times for which determination for setting of a second set of image data has been performed is equal to or less than a threshold that is a preset number of times. If the determination unit 231 determines that the number of times of determination is greater than the threshold (Step S307: No), the processing is returned to Step S301 and the processing is redone from the setting of the first set of image data. On the contrary, if the determination unit 231 determines that the number of times of determination is equal to or less than the threshold (Step S307: Yes), the control unit 23 proceeds to Step S308.

At Step S308, the control unit 23 increments the counter m by 1 and returns to Step S305.

On the contrary, at Step S306, if the determination unit 231 determines that the representative value of the R-pixels is equal to or greater than the threshold (Step S306: Yes), the processing is advanced to Step S309.

At Step S309, the determination unit 231 sets this m-th set of image data as the second set of image data.

At Steps S310 to S313 subsequent to Step S309, the operation control unit 232 calculates the degree of similarity and sets a frame rate, similarly to Steps S110 to S113 illustrated in FIG. 4.

After setting of the frame rate by the operation control unit 232, the control unit 23 determines whether or not a new set of image data has been generated (Step S314). If the control unit 23 determines that a new set of image data has been generated (Step S314: Yes), the control unit 23 returns to Step S301 and repeats the above described processing by treating the latest set of image data at that time point as the n-th set of image data. On the contrary, if a new set of image data has not been generated (Step S314: No), the control unit 23 ends the image data acquisition processing.

According to this third modified example described above, effects similar to those of the first embodiment described above are able to be achieved, and additionally, sets of image data with many residues and/or bubbles are able to be eliminated from targets to be used in control because determination of whether or not an image is suitable for control of the frame rate is done by use of R-pixels. Similar effects are able to be achieved also when B-pixels are used instead of R-pixels.

Fourth Modified Example of First Embodiment

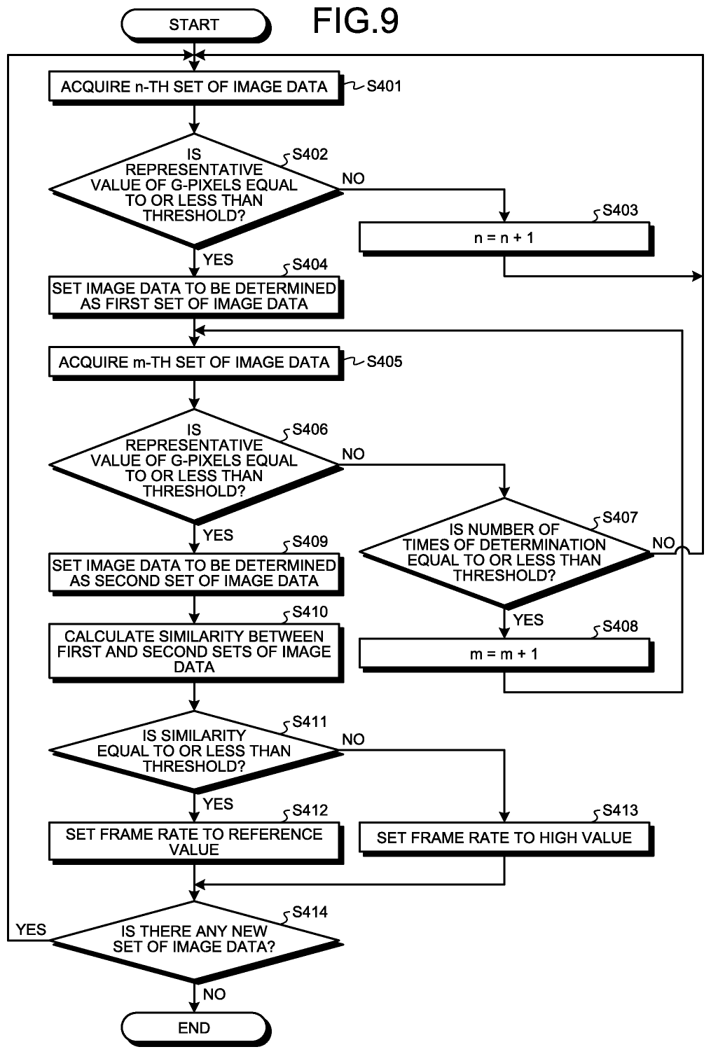

Described next is a fourth modified example of the first embodiment of the present disclosure. FIG. 9 is a flow chart illustrating image data acquisition processing performed by a capsule endoscope system according to a fourth modified example of the first embodiment of the present disclosure. The capsule endoscope system according to the fourth modified example is similar to the capsule endoscope system 1 described above, and each pixel of an imaging element thereof is provided with a color filter, similarly to the third modified example. Described hereinafter by reference to FIG. 9 is processing different from the processing according to the first embodiment described above.

The control unit 23 causes the imaging unit 21 to start imaging processing and acquires an n-th set of image data (Step S401).

At Step S402 subsequent to Step S401, the determination unit 231 calculates a representative value of pixel values of G-pixels from the n-th set of image data acquired at Step S401 and determines whether or not this representative value of the G-pixels is equal to or less than a threshold. The representative value of the G-pixels is, for example, the average value or the total of the pixel values of the G-pixels. If the control unit 23 determines that the representative value of the G-pixels is greater than the threshold (Step S402: No), the control unit 23 proceeds to Step S403.

At Step S403, the control unit 23 increments the counter n by 1 and returns to Step S401.

On the contrary, at Step S402, if the determination unit 231 determines that the representative value of the G-pixels is equal to or less than the threshold (Step S402: Yes), the processing is advanced to Step S404.

At Step S404, the determination unit 231 sets this n-th set of image data as a first set of image data.

At Step S405 subsequent to Step S404, the control unit 23 acquires an m-th set of image data (where m>n).

At Steps S406 to S409 subsequent to Step S405, the determination unit 231 determines whether or not a representative value of G-pixels in the m-th set of image data acquired at Step S405 is equal to or less than the threshold. If the determination unit 231 determines that the representative value of the G-pixels is greater than the threshold (Step S406: No), the control unit 23 proceeds to Step S407.

At Step S407, the determination unit 231 determines whether or not the number of times for which the determination for setting of a second set of image data has been performed is equal to or less than a threshold that is a preset number of times. If the determination unit 231 determines that the number of times of determination is greater than the threshold (Step S407: No), the processing is returned to Step S401 and the processing is redone from the setting of the first set of image data. On the contrary, if the determination unit 231 determines that the number of times of determination is equal to or less than the threshold (Step S407: Yes), the control unit 23 proceeds to Step S408.

At Step S408, the control unit 23 increments the counter m by 1 and returns to Step S405.

On the contrary, at Step S406, if the determination unit 231 determines that the representative value of the G-pixels is equal to or less than the threshold (Step S406: Yes), the processing is advanced to Step S409.

At Step S409, the determination unit 231 sets this m-th set of image data as the second set of image data.

At Steps S410 to S413 subsequent to Step S409, the operation control unit 232 calculates the degree of similarity and sets a frame rate, similarly to Steps S110 to S113 illustrated in FIG. 4.

After setting of the frame rate by the operation control unit 232, the control unit 23 determines whether or not a new set of image data has been generated (Step S414). If the control unit 23 determines that a new set of image data has been generated (Step S414: Yes), the control unit 23 returns to Step S401 and repeats the above described processing by treating the latest set of image data at that time point as the n-th set of image data. On the contrary, if a new set of image data has not been generated (Step S414: No), the control unit 23 ends the image data acquisition processing.

According to the fourth modified example described above, effects similar to those according to the first embodiment described above are able to be achieved, and additionally, brightnesses of images are able to be screened and images having blown out highlights generated therein are able to be eliminated from targets to be used in control because whether or not images are suitable for use in control is determined by use of G-pixels.

According to the above description of the third and fourth modified examples, the average value of pixel values is used as a representative value of R-pixels or G-pixels, but the number of R-pixels or G-pixels having pixel values equal to or greater than a preset value may be used as the representative value.

### Second Embodiment

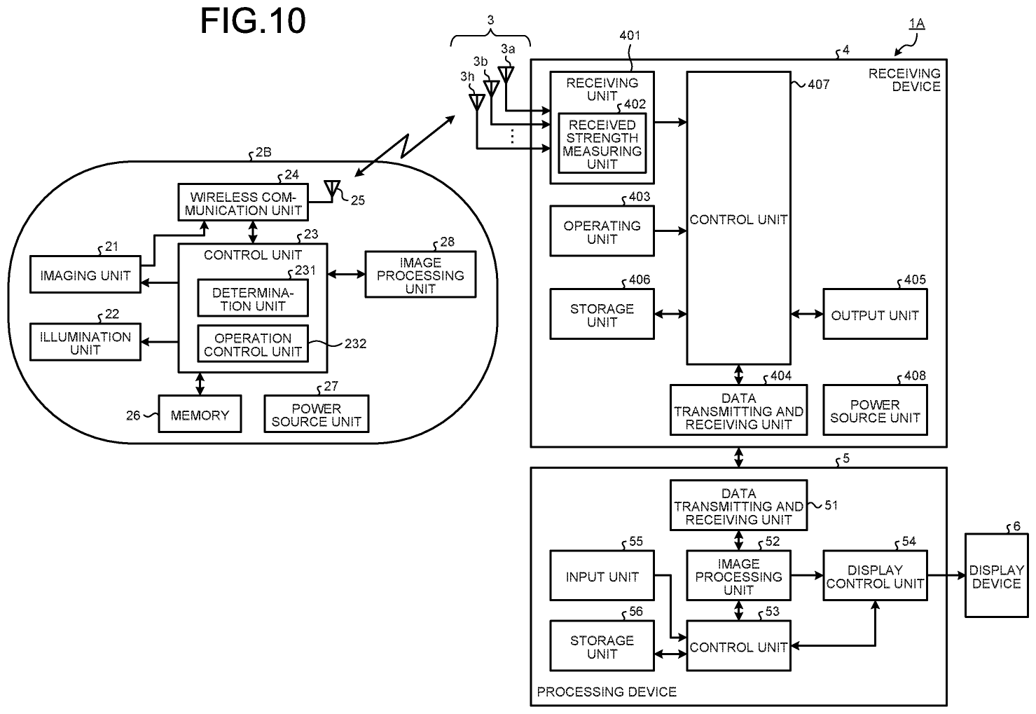

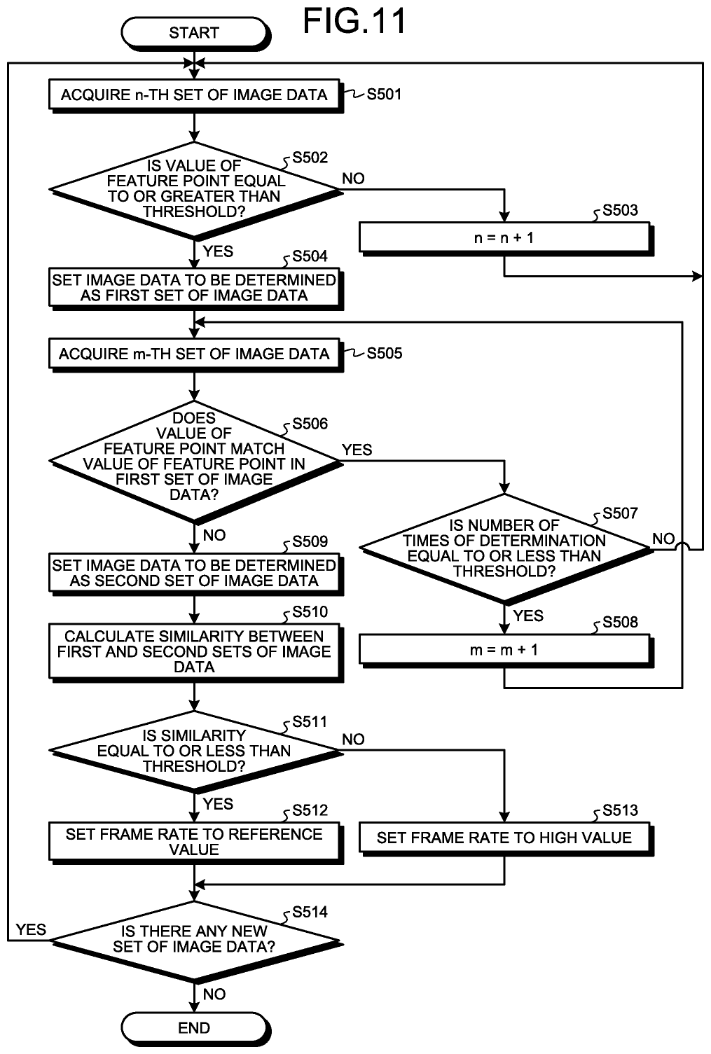

Described next is a second embodiment of the present disclosure. FIG. 10 is a block diagram illustrating a schematic configuration of a capsule endoscope system according to the second embodiment of the present disclosure. A capsule endoscope system 1A according to the second embodiment is different from the capsule endoscope system 1 described above in that the capsule endoscope system 1A includes a capsule endoscope 2B instead of the capsule endoscope 2. The rest of the configuration of the capsule endoscope system 1A is similar to that of the capsule endoscope system 1. Described hereinafter by reference to FIG. 10 and FIG. 11 are the configuration and processing different from the configuration and processing according to the first embodiment described above.

The capsule endoscope 2B includes an imaging unit 21, an illumination unit 22, a control unit 23, a wireless communication unit 24, an antenna 25, a memory 26, a power source unit 27, and an image processing unit 28. That is, the capsule endoscope 2B is different from the capsule endoscope 2 in that the capsule endoscope 2B further includes the image processing unit 28. Described hereinafter is only the image processing unit 28, which is a component different from any of those of the capsule endoscope 2.

By reading a predetermined program stored in the memory 26, the image processing unit 28 performs predetermined image processing for generating an image corresponding to a set of image data generated by the imaging unit 21. Furthermore, by using pixel values (luminance values) of the set of image data that has been image-processed, the image processing unit 28 extracts a features point in the set of image data and calculates a value representing this feature point. Examples of the feature point include: an area of a collection of pixels having a local maximum value or a local minimum value; and an area of a collection of pixels having luminance values that are largely different from luminance values of surrounding pixels. The value representing the feature point is, for example, the average value or the mode value, of luminance values included in the feature point. The value representing the feature point may be a value representing the area of the feature point, such as the number of pixels. The image processing unit 28 is realized by a processor, such as a CPU or an ASIC. The feature point may be extracted based on lightness or hue, instead of luminance.

Described next is image data acquisition processing executed by the capsule endoscope 2B. FIG. 11 is a flow chart illustrating the image data acquisition processing performed by the capsule endoscope system according to the second embodiment of the present disclosure. Hereinafter, description will be made on the assumption that each unit operates under control of the control unit 23.

The control unit 23 causes the imaging unit 21 to start imaging processing and acquires an n-th set of image data (Step S501).

At Step S502 subsequent to Step S501, after extracting a feature point from the n-th set of image data acquired at Step S501 and calculating a value of the feature point, the determination unit 231 determines whether or not this value of the feature point is equal to or greater than a threshold. If the determination unit 231 determines that the value of the feature point is less than the threshold (Step S502: No), the control unit 23 proceeds to Step S503.

At Step S503, the control unit 23 increments the counter n by 1 and returns to Step S501.

On the contrary, at Step S502, if the determination unit 231 determines that the value of the feature point is equal to or greater than the threshold (Step S502: Yes), the processing is advanced to Step S504.

At Step S504, the determination unit 231 sets this n-th set of image data as a first set of image data.

At Step S505 subsequent to Step S504, the control unit 23 acquires an m-th set of image data (where m>n).

At Steps S506 to S509 subsequent to Step S505, the determination unit 231 determines whether or not a value of a feature point in the m-th set of image data acquired at Step S505 matches the value of the feature point in the first set of image data. If the determination unit 231 determines that this value of the feature point matches the value of the feature point in the first set of image data (Step S506: No), the control unit 23 proceeds to Step S507. If the value of the feature point of the m-th set of image data matches the value of the feature point in the first set of image data, the m-th set of image data is able to be determined to be corresponding to an image resulting from just simple rotation of the capsule about an optical axis of the imaging unit 21, the rotation being relative to the image corresponding to the first set of image data. To “match” may mean being in a range of ±several % around the value of the feature point in the first set of image data.

At Step S507, the determination unit 231 determines whether or not the number of times for which determination for setting of a second set of image data has been performed is equal to or less than a threshold that is a preset number of times. If the determination unit 231 determines that the number of times of determination is greater than the threshold (Step S507: No), the processing is returned to Step S501 and the processing is redone from the setting of the first set of image data. On the contrary, if the determination unit 231 determines that the number of times of determination is equal to or less than the threshold (Step S507: Yes), the control unit 23 proceeds to Step S508.

At Step S508, the control unit 23 increments the counter m by 1 and returns to Step S505.

On the contrary, at Step S506, if the determination unit 231 determines that the value of the feature point is different from the value of the feature point in the first set of image data (Step S506: Yes), the processing is advanced to Step S509.

At Step S509, the determination unit 231 sets this m-th set of image data as the second set of image data.

At Steps S510 to S513 subsequent to Step S509, the operation control unit 232 calculates the degree of similarity and sets a frame rate, similarly to Steps S110 to S113 illustrated in FIG. 4. The degree of similarity may be calculated by use of luminance values of the entire sets of image data, or calculated by use of just luminance values of the feature points.

After setting of the frame rate by the operation control unit 232, the control unit 23 determines whether or not a new set of image data has been generated (Step S514). If the control unit 23 determines that a new set of image data has been generated (Step S514: Yes), the control unit 23 returns to Step S501 and repeats the above described processing by treating the latest set of image data at that time point as the n-th set of image data. On the contrary, if a new set of image data has not been generated (Step S514: No), the control unit 23 ends the image data acquisition processing.

According to the second embodiment described above, effects similar to those of the first embodiment described above are able to be achieved, and additionally, an image resulting from just simple rotation of the capsule about the optical axis of the imaging unit 21 is able to be eliminated from targets to be used in control because whether or not the image is suitable for use in control of the frame rate is determined by use of a feature point therein.

### Third Embodiment

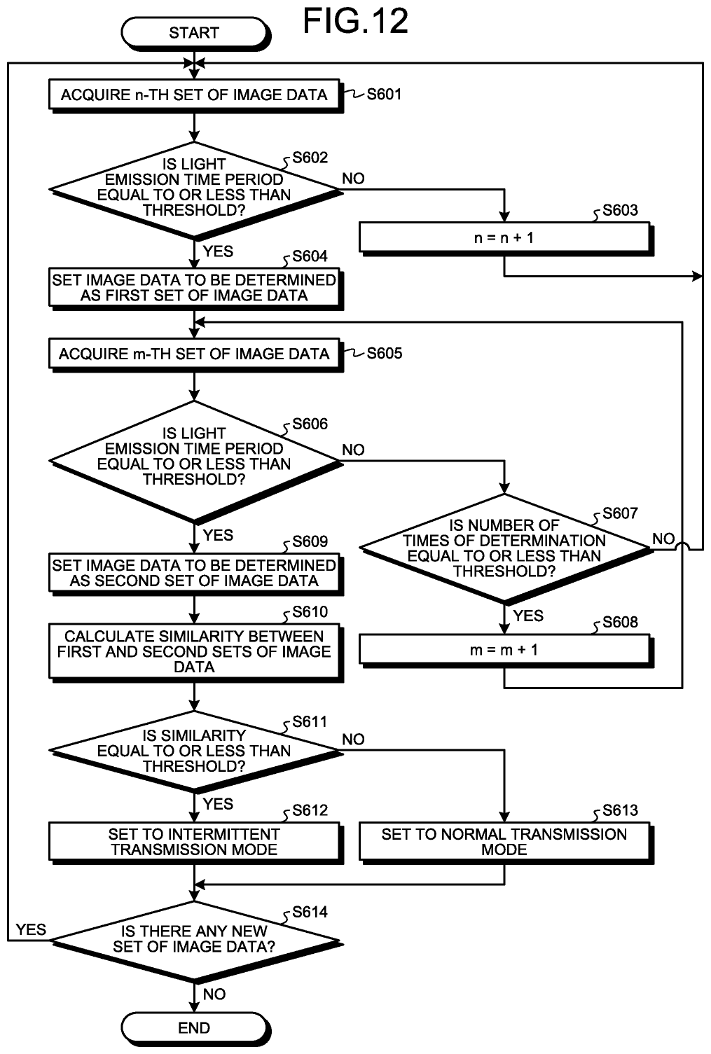

Described next is a third embodiment of the present disclosure. FIG. 12 is a flow chart illustrating image data acquisition processing performed by a capsule endoscope system according to the third embodiment of the present disclosure. The capsule endoscope system according to the third embodiment is similar to the capsule endoscope system 1 described above. Described hereinafter by reference to FIG. 12 is processing different from that according to the first embodiment described above. According to the third embodiment, wireless signal transmission processing is controlled, instead of frame rate, based on image data.

According to the third embodiment, similarly to Steps S101 to S104 illustrated in FIG. 4, the control unit 23 sets an n-th set of image data as a first set of image data (Steps S601 to S604).

Thereafter, similarly to Steps S105 to S109 illustrated in FIG. 4, an m-th set of image data is set as a second set of image data (Steps S605 to S609).

At Step S610 subsequent to Step S609, the operation control unit 232 calculates the degree of similarity, similarly to Step S110 illustrated in FIG. 4.

At Step S611 subsequent to Step S610, the operation control unit 232 determines whether or not the degree of similarity calculated is equal to or less than a threshold. If the operation control unit 232 determines that the degree of similarity is equal to or less than the threshold (Step S611: Yes), the processing is advanced to Step S612.

At Step S612, the operation control unit 232 sets the wireless signal transmission mode to intermittent transmission mode. When the operation control unit 232 sets the transmission mode to the intermittent transmission mode, the operation control unit 232 performs control of thinning out image data generated by the imaging unit 21 and wirelessly transmitting the image data thinned out. As a result, transmission processing with the amount of transmitted image data reduced is performed for similar image data having similar subject images therein.

On the contrary, at Step S611, if the operation control unit 232 determines that the degree of similarity is greater than the threshold (Step S611: No), the processing is advanced to Step S613.

At Step S613, the operation control unit 232 sets the wireless signal transmission mode to normal transmission mode. When the operation control unit 232 sets the transmission mode to the normal transmission mode, the operation control unit 232 performs control of sequentially transmitting image data generated by the imaging unit 21 wirelessly without thinning out the image data. As a result, for image data low in similarity with different subject images therein, the image data generated are sequentially transmitted.

After setting of the transmission mode by the operation control unit 232, the control unit 23 determines whether or not a new set of image data has been generated (Step S614). If the control unit 23 determines that a new set of image data has been generated (Step S614: Yes), the control unit 23 returns to Step S601 and repeats the above described processing by treating the latest set of image data at that time point as the n-th set of image data. On the contrary, if a new set of image data has not been generated (Step S614: No), the control unit 23 ends the image data acquisition processing.

According to the third embodiment described above, whether or not an image is suitable as an image used in setting of the transmission mode is determined from a light emission time period of illumination light for capturing of that set of image data, the degree of similarity between two images determined to be suitable is calculated, and wireless transmission is controlled based on a result of the calculation. According to the third embodiment, because operation control is performed by use of images suitable for use in control, the suitable images being from image data captured by a capsule endoscope, operation of the capsule endoscope is able to be controlled adequately. Furthermore, according to the third embodiment, by wireless transmission of data thinned out, consumption at the power source unit 27 is able to be reduced.

### Fourth Embodiment

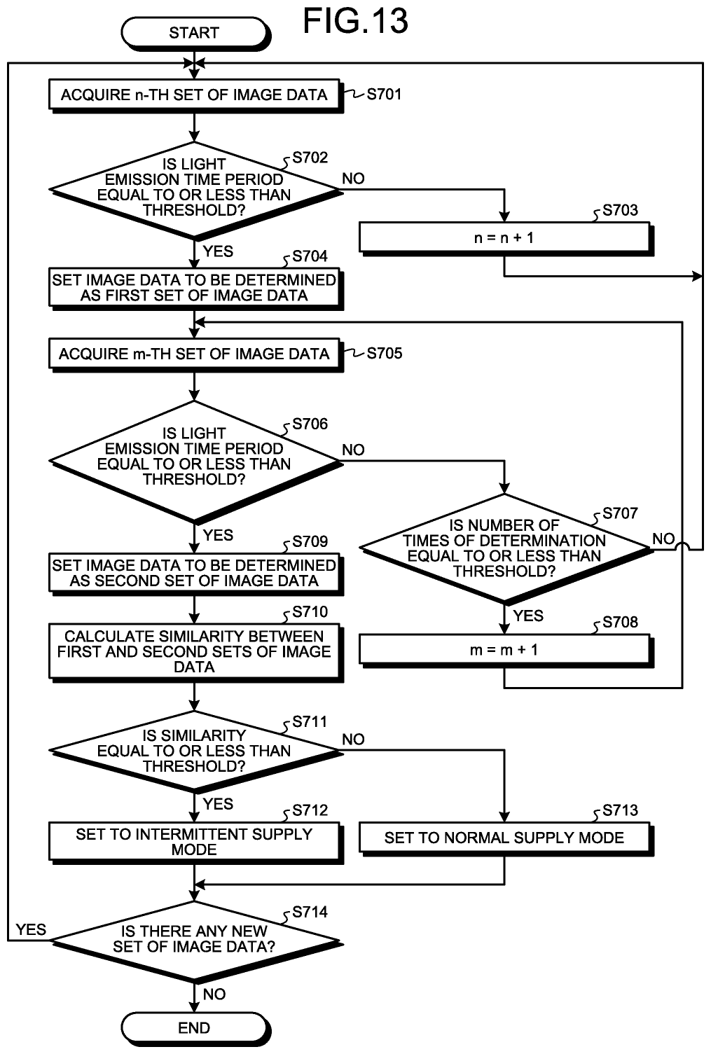

Described next is a fourth embodiment of the present disclosure. FIG. 13 is a flow chart illustrating image data acquisition processing performed by a capsule endoscope system according to the fourth embodiment of the present disclosure. The capsule endoscope system according to the fourth embodiment is similar to the capsule endoscope system 1 described above. Described hereinafter by reference to FIG. 13 is processing different from that according to the first embodiment described above. According to the fourth embodiment, electric power in the capsule endoscope 2 is controlled instead of the frame rate, based on image data.

According to the fourth embodiment, similarly to Steps S101 to S104 illustrated in FIG. 4, the control unit 23 sets an n-th set of image data as a first set of image data (Steps S701 to S704).

Thereafter, similarly to Steps S105 to S109 illustrated in FIG. 4, an m-th set of image data is set as a second set of image data (Steps S705 to S709).

At Step S710 subsequent to Step S709, the operation control unit 232 calculates the degree of similarity, similarly to Step S110 illustrated in FIG. 4.

At Step S711 subsequent to Step S710, the operation control unit 232 determines whether or not the degree of similarity calculated is equal to or less than a threshold. If the operation control unit 232 determines that the degree of similarity is equal to or less than the threshold (Step S711: Yes), the processing is advanced to Step S712.

At Step S712, the operation control unit 232 sets the power supply mode in the capsule endoscope 2 to intermittent supply mode. When the operation control unit 232 sets the power supply mode to the intermittent supply mode, the operation control unit 232 performs control where power supply is intermittently stopped at preset intervals and generation of image data by the imaging unit 21 and wireless communication by the wireless communication unit 24 are intermittently stopped. In the intermittent supply mode, power supply to at least one selected from the group including the imaging unit 21, the illumination unit 22, and the wireless communication unit 24 is controlled. As a result, the amount of image data generated and transmitted for similar images with similar subject images therein is reduced.

On the contrary, at Step S711, if the operation control unit 232 determines that the degree of similarity is greater than the threshold (Step S711: No), the processing is advanced to Step S713.

At Step S713, the operation control unit 232 sets the power supply mode to normal supply mode. When the operation control unit 232 sets the power supply mode to the normal supply mode, the operation control unit 232 perform control of causing the imaging unit 21 to continuously execute generation of image data and the wireless communication unit 24 to continuously execute wireless communication without causing them to stop the generation and the wireless communication. As a result, for images that are low in similarity and have different subject images therein, image data corresponding to the images are sequentially transmitted.

After setting of the power supply mode by the operation control unit 232, the control unit 23 determines whether or not a new set of image data has been generated (Step S714). If the control unit 23 determines that a new set of image data has been generated (Step S714: Yes), the control unit 23 returns to Step S701 and repeats the above described processing by treating the latest set of image data at that time point as the n-th set of image data. On the contrary, if a new set of image data has not been generated (Step S714: No), the control unit 23 ends the image data acquisition processing.

According to the fourth embodiment described above, whether or not an image is suitable for use in setting of the power supply mode is determined from a light emission time period of illumination light for capturing of this set of image data, the degree of similarity between two images determined to be suitable is calculated, and power supply is controlled based on a result of the calculation. According to the fourth embodiment, because operation control is performed by use of image data suitable for use in control, the image data being from image data captured by a capsule endoscope, operation of the capsule endoscope is able to be controlled adequately. Furthermore, according to the fourth embodiment, by intermittent power supply, consumption at the power source unit 27 is able to be reduced.

### Fifth Embodiment

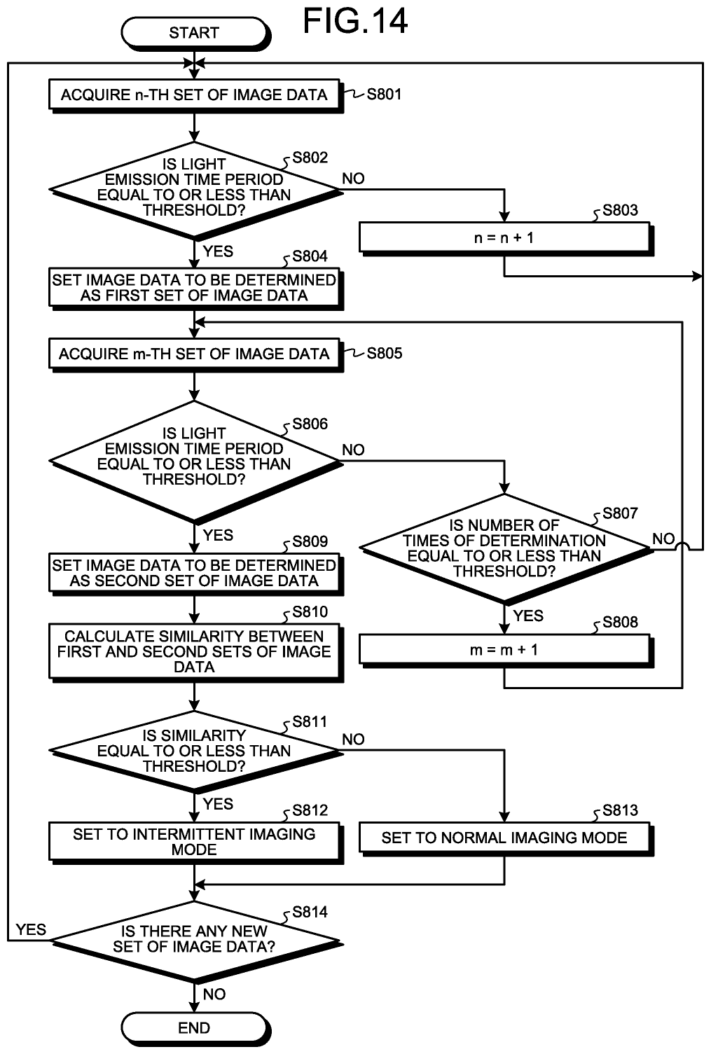

Described next is a fifth embodiment of the present disclosure. FIG. 14 is a flow chart illustrating image data acquisition processing performed by a capsule endoscope system according to the fifth embodiment of the present disclosure. The capsule endoscope system according to the fifth embodiment is similar to the capsule endoscope system 1 described above. Described hereinafter by reference to FIG. 14 is processing different from that according to the first embodiment described above. According to the fifth embodiment, based on image data, control of whether or not imaging processing itself is to be temporarily stopped is performed instead of setting of the frame rate.

According to the fifth embodiment, similarly to Steps S101 to S104 illustrated in FIG. 4, the control unit 23 sets an n-th set of image data as a first set of image data (Steps S801 to S804).

Thereafter, similarly to Steps S105 to S109 illustrated in FIG. 4, an m-th set of image data is set as a second set of image data (Steps S805 to S809).

At Step S810 subsequent to Step S809, the operation control unit 232 calculates the degree of similarity, similarly to Step S110 illustrated in FIG. 4.

At Step S811 subsequent to Step S810, the operation control unit 232 determines whether or not the degree of similarity calculated is equal to or less than a threshold. If the operation control unit 232 determines that the degree of similarity is equal to or less than the threshold (Step S811: Yes), the processing is advanced to Step S812.

At Step S812, the operation control unit 232 sets the imaging mode of the imaging unit 21 to intermittent imaging mode. When the operation control unit 232 sets the imaging mode to the intermittent imaging mode, the operation control unit 232 performs control where imaging processing corresponding to a present number of frames is stopped and generation of image data by the imaging unit 21 and wireless communication by the wireless communication unit 24 are intermittently stopped. As a result, the amount of image data generated and transmitted for similar images with similar subject images therein is reduced.

On the contrary, at Step S811, if the operation control unit 232 determines that the degree of similarity is greater than the threshold (Step S811: No), the processing is advanced to Step S813.

At Step S813, the operation control unit 232 sets the imaging mode to normal imaging mode. When the operation control unit 232 sets the imaging mode to the normal imaging mode, the operation control unit 232 performs control of causing the imaging unit 21 to continuously execute generation of image data and the wireless communication unit 24 to continuously execute wireless communication without causing them to stop the generation and the wireless communication. As a result, image data corresponding to images that are low in similarity and have different subject images therein are sequentially transmitted.

After setting of the imaging mode by the operation control unit 232, the control unit 23 determines whether or not a new set of image data has been generated (Step S814). If the control unit 23 determines that a new set of image data has been generated (Step S814: Yes), the control unit 23 returns to Step S801 and repeats the above described processing by treating the latest set of image data at that time point as the n-th set of image data. On the contrary, if a new set of image data has not been generated (Step S814: No), the control unit 23 ends the image data acquisition processing.

According to the fifth embodiment described above, whether or not an image is suitable for use in setting of the imaging mode is determined from a light emission time period of illumination light for capturing of this set of image data, the degree of similarity between two images determined to be suitable is calculated, and imaging processing is controlled based on a result of the calculation. According to the fifth embodiment, because operation control is performed by use of image data suitable for use in control, the image data being from image data captured by a capsule endoscope, operation of the capsule endoscope is able to be controlled adequately. Furthermore, according to the fifth embodiment, by intermittent imaging processing, consumption at the power source unit 27 is able to be reduced.

According to the fifth embodiment described above, instead of control of imaging operation by the imaging unit 21, control of emission of illumination light by the illumination unit 22 may be performed. For example, if the imaging mode has been set to the intermittent imaging mode, intensity of illumination light from the illumination unit 22 may be reduced or the number of times of emission per unit time may be controlled.

### Sixth Embodiment

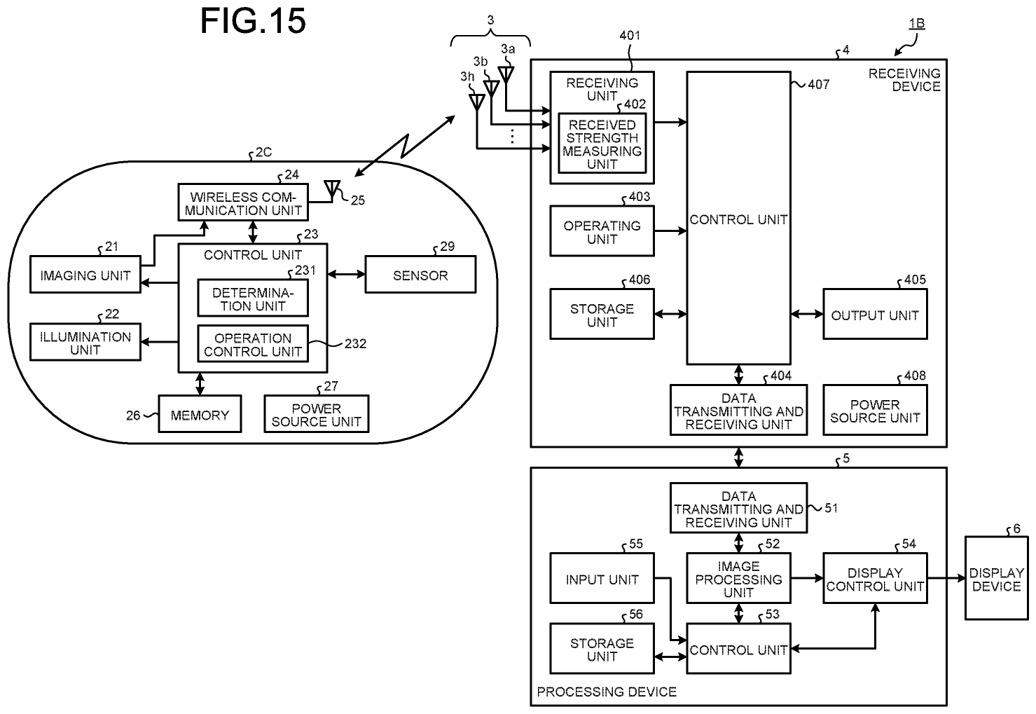

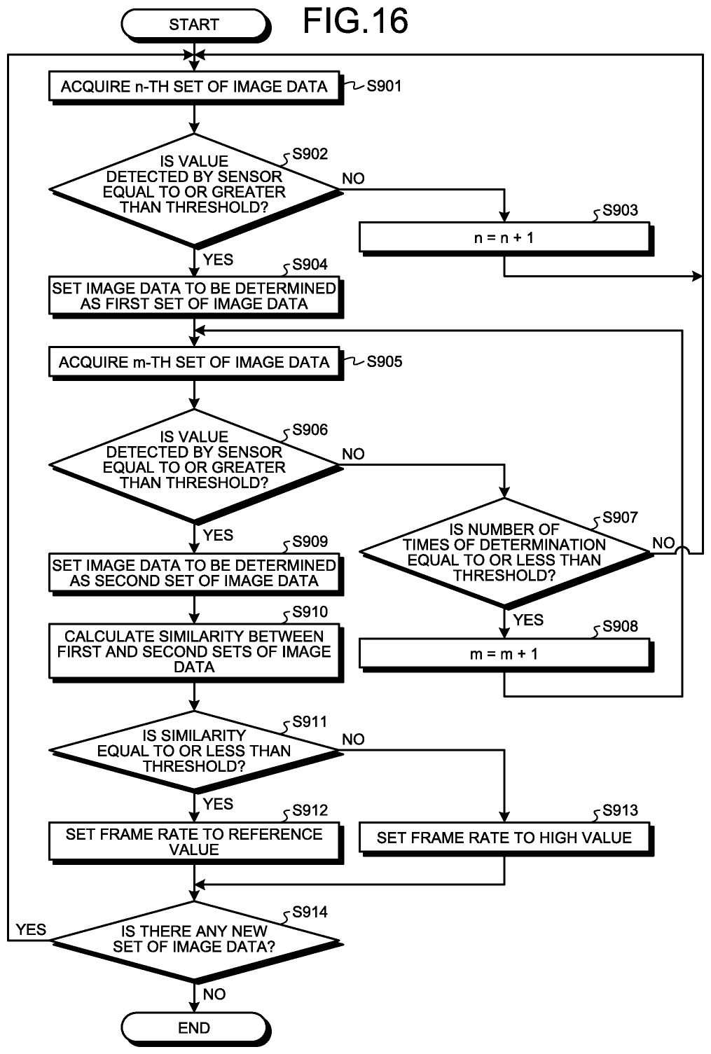

Described next is a sixth embodiment of the present disclosure. FIG. 15 is a block diagram illustrating a schematic configuration of a capsule endoscope system according to the sixth embodiment of the present disclosure. A capsule endoscope system 1B according to the sixth embodiment is different from the capsule endoscope system 1 described above in that the capsule endoscope system 1B includes a capsule endoscope 2C instead of the capsule endoscope 2. The rest of the configuration of the capsule endoscope system 1B is similar to that of the capsule endoscope system 1. Described hereinafter by reference to FIG. 15 and FIG. 16 are the configuration and processing different from those according to the first embodiment described above.

The capsule endoscope 2C includes an imaging unit 21, an illumination unit 22, a control unit 23, a wireless communication unit 24, an antenna 25, a memory 26, a power source unit 27, and a sensor 29. That is, the capsule endoscope 2C is different from the capsule endoscope 2 in that the capsule endoscope 2C further includes the sensor 29. Described hereinafter is only the sensor 29, which is the part different from the capsule endoscope 2.

The sensor 29 is a pressure sensor that detects pressure applied to the capsule endoscope 2C. The sensor 29 outputs a detected value that is a value corresponding to an amount of deformation of a diaphragm deformed according to a load from outside. The sensor 29 detects, for example, a change in electrostatic capacity that changes according to deformation of a diaphragm, converts this change into a detected value, and outputs the detected value. The sensor 29 may be, instead of the pressure sensor, an acceleration sensor, an earth magnetism sensor, or a pH sensor.

Described next is image data acquisition processing executed by the capsule endoscope 2C. FIG. 16 is a flow chart illustrating image data acquisition processing performed by the capsule endoscope system according to the sixth embodiment of the present disclosure. Hereinafter, description will be made on the assumption that each unit operates under control of the control unit 23.

The control unit 23 causes the imaging unit 21 to start imaging processing and acquires an n-th set of image data (Step S901). According to the sixth embodiment, a detected value in the sensor 29 detected at the time of imaging has been assigned to each set of image data.

At Step S902 subsequent to Step S901, the determination unit 231 extracts the detected value in the sensor 29 from the n-th set of image data acquired at Step S901 and determines whether or not this detected value is equal to or greater than a threshold. If the determination unit 231 determines that the detected value is less than the threshold (Step S902: No), the control unit 23 proceeds to Step S903. If the detected value is less than the threshold, space where the capsule endoscope 2C is present is able to be determined to be wide. A part having wide space in the subject H is, for example, the stomach.

At Step S903, the control unit 23 increments the counter n by 1 and returns to Step S901.

On the contrary, at Step S902, if the determination unit 231 determines that the detected value is equal to or greater than the threshold (Step S902: Yes), the processing is advanced to Step S904. If the detected value is equal to or greater than the threshold, space where the capsule endoscope 2C is present is able to be determined to be narrow. A part having narrow space in the subject H is, for example, the small intestine.

At Step S904, the determination unit 231 sets this n-th set of image data as a first set of image data.

At Step S905 subsequent to Step S904, the control unit 23 acquires an m-th set of image data (where m>n).

At Steps S906 to s909 subsequent to Step S905, the determination unit 231 determines whether or not a detected value by the sensor 29 in the m-th set of image data acquired at Step S905 is equal to or greater than the threshold. If the determination unit 231 determines that the detected value is less than the threshold (Step S906: No), the control unit 23 proceeds to Step S907.

At Step S907, the determination unit 231 determines whether or not the number of times for which determination for setting of a second set of image data has been performed is equal to or less than a threshold that is a preset number of times. If the determination unit 231 determines that the number of times of determination is greater than the threshold (Step S907: No), the processing is returned to Step S901 and the processing is redone from the setting of the first set of image data. On the contrary, if the determination unit 231 determines that the number of times of determination is equal to or less than the threshold (Step S907: Yes), the control unit 23 proceeds to Step S908.

At Step S908, the control unit 23 increments the counter m by 1 and returns to Step S905.

On the contrary, at Step S906, if the determination unit 231 determines that the detected value is equal to or greater than the threshold (Step S906: Yes), the processing is advanced to Step S909.

At Step S909, the determination unit 231 sets this m-th set of image data as a second set of image data.

At Steps S910 to S913 subsequent to Step S909, the operation control unit 232 calculates the degree of similarity and sets a frame rate, similarly to Steps S110 to S113 illustrated in FIG. 4.

After setting of the frame rate by the operation control unit 232, the control unit 23 determines whether or not a new set of image data has been generated (Step S914). If the control unit 23 determines that a new set of image data has been generated (Step S914: Yes), the control unit 23 returns to Step S901 and repeats the above described processing by treating the latest set of image data at that time point as the n-th set of image data. On the contrary, if a new set of image data has not been generated (Step S914: No), the control unit 23 ends the image data acquisition processing.

According to the above described sixth embodiment, the frame rate is set based on a result of detection by the sensor 29 at the time of capturing of image data. According to the sixth embodiment, operation of a capsule endoscope is able to be controlled adequately because operation control is performed according to the external environment of the capsule endoscope.

According to the sixth embodiment of the present disclosure, a difference between the detected value by the sensor for the n-th set of image data and the detected value by the sensor for the (n+1)-th set of image data is calculated, comparison between the calculated difference and a threshold is performed, a change in space where the capsule endoscope 2 is present is thereby detected, and whether or not these sets of image data are to be used for setting of the frame rate may thereby be determined.

### Seventh Embodiment

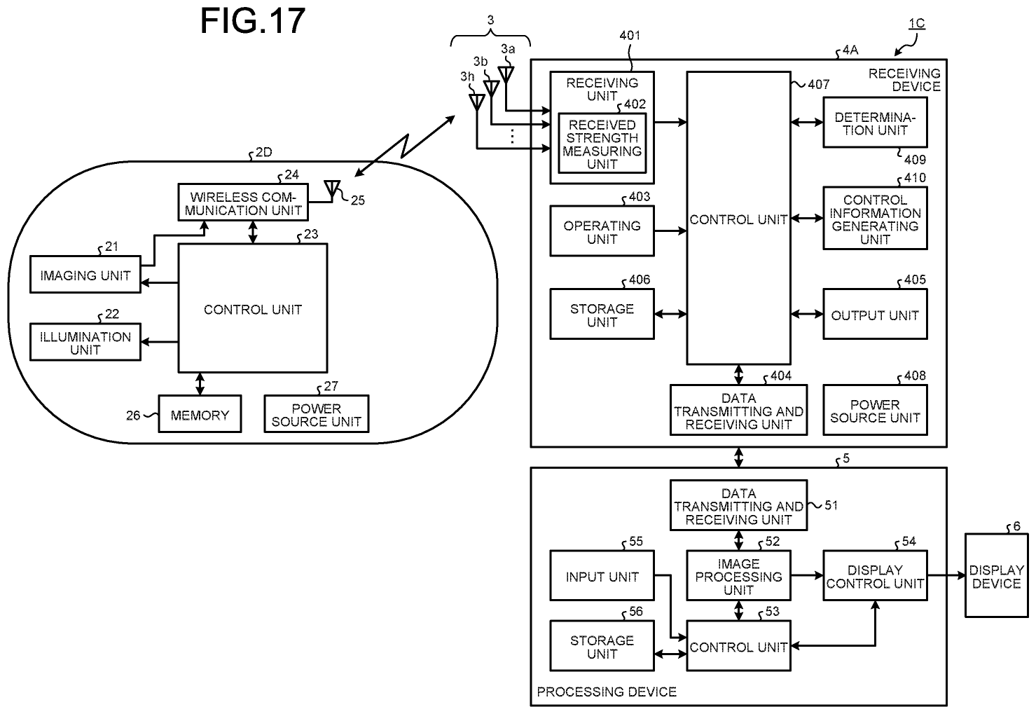

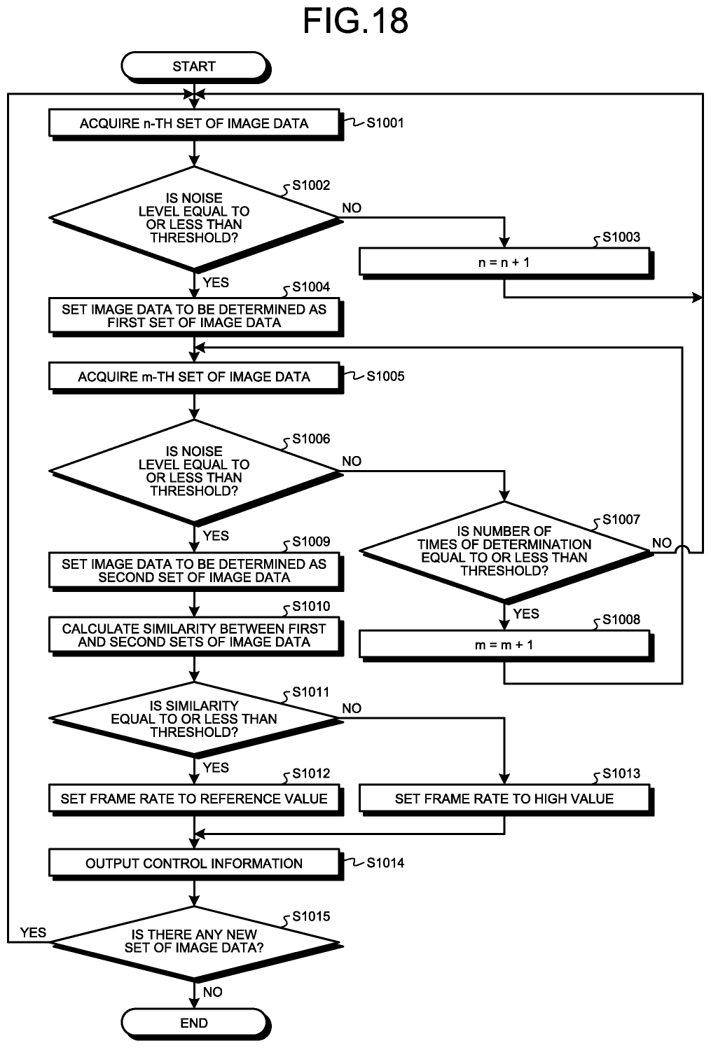

Described next is a seventh embodiment of the present disclosure. FIG. 17 is a block diagram illustrating a schematic configuration of a capsule endoscope system according to the seventh embodiment of the present disclosure. A capsule endoscope system 1C according to the seventh embodiment is different from the capsule endoscope system 1 described above in that the capsule endoscope system 1C includes a capsule endoscope 2D instead of the capsule endoscope 2 and includes a receiving device 4A instead of the receiving device 4. The rest of the configuration of the capsule endoscope system 1C is similar to that of the capsule endoscope system 1. Described hereinafter by reference to FIG. 17 and FIG. 18 are the configuration and processing different from those according to the first embodiment described above.

The capsule endoscope 2D includes an imaging unit 21, an illumination unit 22, a control unit 23, a wireless communication unit 24, an antenna 25, a memory 26, and a power source unit 27. Furthermore, the control unit 23 does not have the determination unit 231 and the operation control unit 232. That is, the capsule endoscope 2D is different from the capsule endoscope 2 in that the capsule endoscope 2D does not have the determination unit 231 and the operation control unit 232.

The receiving device 4A includes a receiving unit 401, a received strength measuring unit 402, an operating unit 403, a data transmitting and receiving unit 404, an output unit 405, a storage unit 406, a control unit 407, a power source unit 408, a determination unit 409, and a control information generating unit 410. That is, the receiving device 4A is different from the receiving device 4 in that the receiving device 4A further includes the determination unit 409 and the control information generating unit 410. Described hereinafter are only the determination unit 409 and the control information generating unit 410 that are parts different from the receiving device 4.

Similarly to the determination unit 231 described above, the determination unit 409 selects two sets of image data by determining, based on noise level of image data received by the receiving device 4A, whether or not the sets of image data are suitable for use in control of frame rate. The determination unit 409 calculates an S/N ratio (dB) from a wireless signal received by the receiving device 4A and determines a noise level that is the reciprocal of this S/N ratio. The determination unit 409 is realized by a processor, such as a CPU or an ASIC.

Similarly to the operation control unit 232 described above, the control information generating unit 410 calculates the degree of similarity between the two images selected by the determination unit 409, and compares this similarity with a threshold stored beforehand in the storage unit 406. The control information generating unit 410 generates control information related to the frame rate of the imaging unit 21, according to a result of the comparison between the degree of similarity and the threshold. The control information generating unit 410 is realized by a processor, such as a CPU or an ASIC.

Described next is image data acquisition processing executed by the capsule endoscope system 1C. FIG. 18 is a flow chart illustrating image data acquisition processing performed by the capsule endoscope system according to the seventh embodiment of the present disclosure.

The control unit 407 of the receiving device 4A causes the imaging unit 21 to start imaging processing and acquires an n-th set of image data (Step S1001).

At Step S1002 subsequent to Step S1001, the determination unit 409 finds a noise level that is the reciprocal of an S/N ratio of a wireless signal for reception of the n-th set of image data acquired at Step S1001, and determines whether this noise level is equal to or less than a threshold. If the determination unit 409 determines that the noise level is greater than the threshold (Step S1002: No), the control unit 407 proceeds to Step S1003.

At Step S1003, the control unit 407 increments the counter n by 1 and returns to Step S1001.

On the contrary, at Step S1002, if the determination unit 409 determines that the noise level is equal to or less than the threshold (Step S1002: Yes), the determination unit 409 proceeds to Step S1004.

At Step S1004, the determination unit 409 sets the n-th set of image data as a first set of image data.

At Step S1005 subsequent to Step S1004, the control unit 407 acquires an m-th set of image data (where m>n).

At Step S1006 subsequent to Step S1005, the determination unit 409 determines whether or not a noise level in the m-th set of image data acquired at Step S1005 is equal to or greater than a threshold. If the determination unit 409 determines that the noise level is greater than the threshold (Step S1006: No), the control unit 407 proceeds to Step S1007.

At Step S1007, the determination unit 409 determines whether or not the number of times for which the determination for setting of a second set of image data has been performed is equal to or less than a threshold that is a preset number of times. If the determination unit 409 determines that the number of times of determination is greater than the threshold (Step S1007: No), the processing is returned to Step S1001 and the processing is redone from the setting of the first set of image data. On the contrary, if the determination unit 409 determines that the number of times of determination is equal to or less than the threshold (Step S1007: Yes), the control unit 407 proceeds to Step S1008.

At Step S1008, the control unit 407 increments the counter m by 1 and returns to Step S1005.

On the contrary, at Step S1006, if the determination unit 409 determines that the noise level is equal to or less than the threshold (Step S1006: Yes), the processing is advanced to Step S1009.

At Step S1009, the determination unit 409 sets this m-th set of image data as the second set of image data.

At Steps S1010 to S1013 subsequent to Step S1009, the control information generating unit 410 calculates the degree of similarity and sets a frame rate, similarly to Steps S110 to S113 illustrated in FIG. 4.

At Step S1014 subsequent to Step S1012 or S1013, the control information generating unit 410 generates control information related to the set frame rate and outputs this control information to the capsule endoscope 2D. Imaging processing in the capsule endoscope 2D is thereby controlled according to the frame rate set in the receiving device 4A.

After the output of the control information by the control information generating unit 410, the control unit 407 determines whether or not a new set of image data has been generated (Step S1015). If the control unit 407 determines that a new set of image data has been generated (Step S1015: Yes), the control unit 407 returns to Step S1001 and repeats the above described processing by treating the latest set of image data at that time point as the n-th set of image data. On the contrary, if a new set of image data has not been generated (Step S1015: No), the control unit 407 ends the image data acquisition processing.

According to the seventh embodiment described above, even if control by the receiving device 4A is performed, effects similar to those of the first embodiment described above are able to be achieved.

According to the seventh embodiment described above, the operation control unit 232 may be provided in the capsule endoscope 2D, a determination result by the determination unit 409 may be transmitted to the capsule endoscope 2D, and imaging control based on the determination result may be performed in the capsule endoscope 2D.

Modified Example of Seventh Embodiment

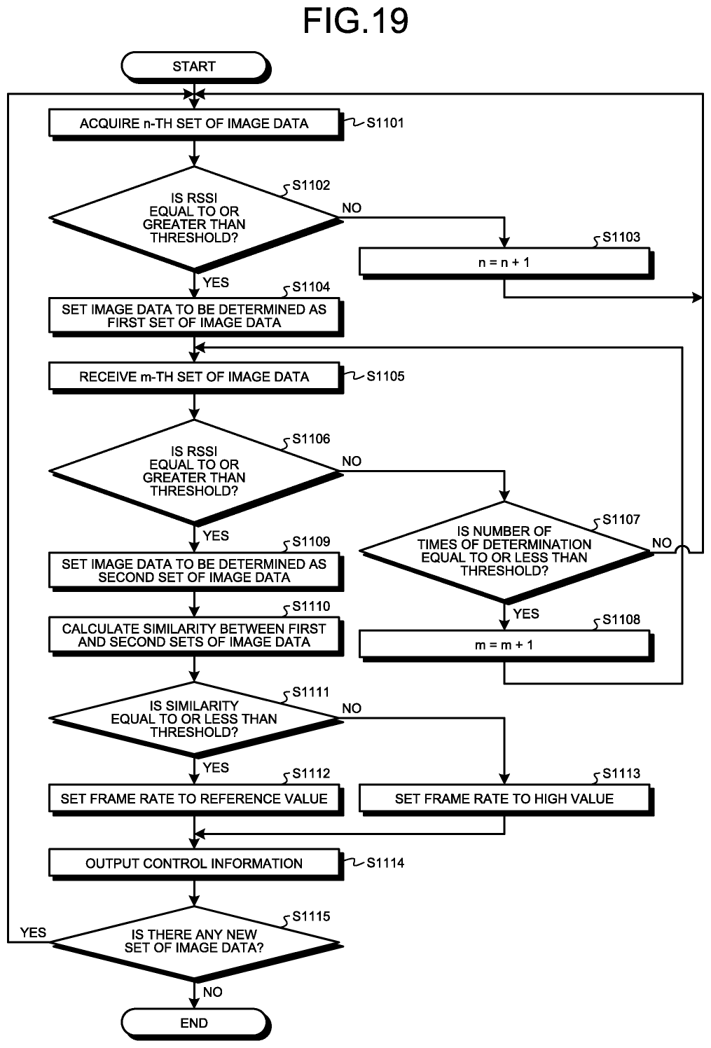

Described next is a modified example of a seventh embodiment of the present disclosure. FIG. 19 is a flow chart illustrating image data acquisition processing performed by a capsule endoscope system according to the modified example of the seventh embodiment of the present disclosure. The capsule endoscope system according to the modified example is similar to the capsule endoscope system 1C described above. Described hereinafter by reference to FIG. 19 is processing different from that according to the seventh embodiment described above.

The control unit 407 causes the imaging unit 21 to start imaging processing and acquires an n-th set of image data (Step S1101).

At Step S1102 subsequent to Step S1101, the determination unit 409 determines whether or not an RSSI corresponding to the n-th set of image data acquired at Step S1101 is equal to or greater than a threshold. If the determination unit 409 determines that the RSSI is less than the threshold (Step S1102: No), the control unit 407 proceeds to Step S1103.

At Step S1103, the control unit 407 increments the counter n by 1 and returns to Step S1201.

On the contrary, at Step S1102, if the determination unit 409 determines that the RSSI is equal to or greater than the threshold (Step S1102: Yes), the processing is advanced to Step S1104.

At Step S1104, the determination unit 409 sets the n-th set of image data as a first set of image data.

At Step S1105 subsequent to Step S1104, the control unit 407 acquires an m-th set of image data (where m>n).

At Step S1106 subsequent to Step S1105, the determination unit 409 determines whether or not an RSSI corresponding to the m-th set of image data acquired at Step S1105 is equal to or greater than a threshold. If the determination unit 409 determines that the RSSI is less than the threshold (Step S1106: No), the control unit 407 proceeds to Step S1107.

At Step S1107, the determination unit 409 determines whether or not the number of times for which determination for setting of a second set of image data has been performed is equal to or less than a threshold that is a preset number of times. If the determination unit 409 determines that the number of times of determination is greater than the threshold (Step S1107: No), the processing is returned to Step S1101 and the processing is redone from the setting of the first set of image data. On the contrary, if the determination unit 409 determines that the number of times of determination is equal to or less than the threshold (Step S1107: Yes), the control unit 407 proceeds to Step S1108.

At Step S1108, the control unit 407 increments the counter m by 1 and returns to Step S1105.

On the contrary, at Step S1106, if the determination unit 409 determines that the RSSI is equal to or greater than the threshold (Step S1106: Yes), the processing is advanced to Step S1109.

At Step S1109, the determination unit 409 sets this m-th set of image data as the second set of image data.

At Steps S1110 to S1114 subsequent to Step S1109, the control information generating unit 410 calculates the degree of similarity, sets a frame rate, and performs output processing for control information, similarly to Steps S1010 to S1014 illustrated in FIG. 18.

After the output of the control information by the control information generating unit 410, the control unit 407 determines whether or not a new set of image data has been generated (Step S1115). If the control unit 407 determines that a new set of image data has been generated (Step S1115: Yes), the control unit 407 returns to Step S1101 and repeats the above described processing by treating the latest set of image data at that time point as the n-th set of image data. On the contrary, if a new set of image data has not been generated (Step S1115: No), the control unit 407 ends the image data acquisition processing.

According to this modified example described above, even if received signal strength indicators (RSSIs) are used, effects similar to those of the seventh embodiment described above are able to be achieved. Instead of the above described noise level or RSSI, the number of flickering noises corresponding to the number of pixels having prominent luminance values or the number of times of error correction may be used.

Thus far, modes for implementation of the present disclosure have been described, but the present disclosure is not to be limited only to the above described embodiments and modified examples. Without being limited to the above described embodiments and modified examples, the present disclosure may include various embodiments without departing from the technical ideas stated in the claims. Furthermore, the components according to the embodiments and modified examples may be combined as appropriate.

Furthermore, the execution program for the processing executed by each component of the capsule endoscopes, receiving devices, and processing devices, of the capsule endoscope systems according to the first to seventh embodiments: may be configured to be provided by being recorded, in a file having an installable format or an executable format, on a computer readable recording medium, such as a CD-ROM, a flexible disk (FD), a CD-R, or a DVD; or may be configured to be provided by being stored on a computer connected to a network, such as the Internet, and being downloaded via the network. Furthermore, the execution program may be configured to be provided or distributed via a network, such as the internet.

Furthermore, if the antenna with the highest received signal strength indicator (RSSI) has not changed, the frame rate may be set to a reference value or a low value as if the existing position of the capsule endoscope has not changed; a set of image data for which a synchronization signal was unable to be received may be not selected as a set of image data to be used in control; or if a synchronization signal has been unable to be received in frames previous and subsequent to a frame, a set of image data corresponding to the frame positioned between these frames may be not selected as a set of image data for use in control.

Furthermore, the above described processing for setting of the frame rate according to the first or second embodiment may be performed in the receiving device.

As described above, a capsule endoscope system and a capsule endoscope according to the present disclosure are useful for adequate control of operation of the capsule endoscope by use of images captured by the capsule endoscope.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A capsule endoscope system, comprising:
a capsule endoscope configured to generate images by imaging a subject through irradiation of the subject with illumination light; and
a processor comprising hardware, the processor being configured to determine, based on an image to be determined or associated information associated with the image to be determined, whether or not the image to be determined is suitable for control of operation of the capsule endoscope, the image being one of the images generated.

2. The capsule endoscope system according to claim 1, wherein the processor is configured to set a first image that has been determined to be suitable and thereafter set a second image from an image group that is chronologically before or after the first image.

3. The capsule endoscope system according to claim 2, wherein the processor is further configured to control operation of the capsule endoscope, based on the first image and the second image that have been determined to be suitable.

4. The capsule endoscope system according to claim 1, wherein the processor is configured to determine, based on imaging information for the imaging of the images, whether or not the image to be determined is suitable as one of a first image and a second image.

5. The capsule endoscope system according to claim 4, wherein the processor is configured to determine, based on light emission quantity of the illumination light for the generation of the images, whether or not the image to be determined is suitable as one of the first image and the second image.

6. The capsule endoscope system according to claim 4, wherein the processor is configured to determine, based on first pixel information of the images, whether or not the image to be determined is suitable as one of the first image and the second image.

7. The capsule endoscope system according to claim 6, wherein the processor is configured to determine, based on second pixel information related to a residue or a bubble, whether or not the image to be determined is suitable as one of the first image and the second image, the second pixel information being from the first pixel information of the images.

8. The capsule endoscope system according to claim 6, wherein the processor is configured to set one of the first image and the second image, and thereafter set, based on presence or absence of rotation about an optical axis of the image to be determined, other one of the first image and the second image, the rotation being relative to the one of the first image and the second image and being detected based on pixel information on the one of the first image and the second image and pixel information on the image to be determined.

9. The capsule endoscope system according to claim 1, wherein
the capsule endoscope includes a sensor configured to detect information on operation or an external environment of the capsule endoscope, and
the processor is configured to determine, based on the information detected by the sensor, whether or not the image to be determined is suitable.

10. The capsule endoscope system according to claim 1, wherein
the capsule endoscope includes a transmitter configured to wirelessly transmit the images generated to outside,
the capsule endoscope system further comprises a receiving device including a receiver configured to receive the images wirelessly transmitted by the transmitter, and
the processor is provided in the receiving device and is configured to determine, based on information on the reception of the images by the receiver, whether or not the image to be determined is suitable.

11. The capsule endoscope system according to claim 3, wherein
the capsule endoscope includes an imager configured to generate the images, and
the processor is configured to control frame rate of the imager, based on the first image or the second image that has been determined to be suitable as an image for controlling the operation of the capsule endoscope.

12. The capsule endoscope system according to claim 3, wherein
the capsule endoscope includes:
an illuminator configured to emit the illumination light; and
an imager configured to generate the images, and the processor is configured to control at least one of imaging operation of the imager and illumination operation of the illuminator, based on the first image or the second image that has been determined to be suitable as an image for controlling the operation of the capsule endoscope.

13. The capsule endoscope system according to claim 3, wherein
the capsule endoscope includes a transmitter configured to wirelessly transmit the images to outside, and
the processor is configured to control transmitting operation of the transmitter, based on the first image or the second image that has been determined to be suitable as an image for controlling the operation of the capsule endoscope.

14. The capsule endoscope system according to claim 3, wherein
the capsule endoscope includes a power source configured to supply electric power to a circuit that is inside the capsule endoscope, and
the processor is configured to control electric power supplying operation of the power source, based on the first image or the second image that has been determined to be suitable as an image for controlling the operation of the capsule endoscope.

15. A capsule endoscope configured to generate images by imaging a subject through irradiation of the subject with illumination light, the capsule endoscope comprising:
a processor comprising hardware, the processor being configured to determine, based on an image to be determined or associated information associated with the image to be determined, whether or not the image to be determined is suitable for control of operation of the capsule endoscope, the image to be determined being one of the images generated.

16. A receiving device configured to receive images wirelessly transmitted by a capsule endoscope configured to generate the images by imaging a subject through irradiation of the subject with illumination light, the receiving device comprising:
a processor comprising hardware, the processor being configured to
determine, based on an image to be determined or associated information associated with the image to be determined, whether or not the image to be determined is suitable for control of operation of the capsule endoscope, the image to be determined being one of the images generated, and
generate, from information obtained from the image that have been determined to be suitable, control information related to operation control of the capsule endoscope.
